SEQUENCE LISTING (87 sequences)

SEQ ID NO: 1 - protein, 10 aa, Artificial Sequence
  Synthetic construct
WWRRVRRRWR

SEQ ID NO: 2 - protein, 12 aa, Artificial Sequence
  Synthetic construct
WWRRVRRRWRRV

SEQ ID NO: 3 - protein, 14 aa, Artificial Sequence
  Synthetic construct
WWRRVRRVWRRVRR

SEQ ID NO: 4 - protein, 16 aa, Artificial Sequence
  Synthetic construct
WWRRVRRVWRRVRRRV

SEQ ID NO: 5 - protein, 18 aa, Artificial Sequence
  Synthetic construct
VWRRVRRVWRRVRRWVRR

SEQ ID NO: 6 - protein, 18 aa, Artificial Sequence
  Synthetic construct
WWRRVRRVWRRVRRVVRR

SEQ ID NO: 7 - protein, 20 aa, Artificial Sequence
  Synthetic construct
RWVRRVRRVWRRVRRVVRRW

SEQ ID NO: 8 - protein, 22 aa, Artificial Sequence
  Synthetic construct
RRWVRRVRRVWRRVRRVVRRWV

SEQ ID NO: 9 - protein, 24 aa, Artificial Sequence
  Synthetic construct
RRVWRRVRRVVRRVWRWVRRVVRR

SEQ ID NO: 10 - protein, 10 aa, Artificial Sequence
  Synthetic construct
RRWWRRVWRW

SEQ ID NO: 11 - protein, 12 aa, Artificial Sequence
  Synthetic construct
RRVWRWVRRWWR

SEQ ID NO: 12 - protein, 12 aa, Artificial Sequence
  Synthetic construct
WWRRWRRRWRRV

SEQ ID NO: 13 - protein, 14 aa, Artificial Sequence
  Synthetic construct
RRVWRWVRRWWRRV

SEQ ID NO: 14 - protein, 16 aa, Artificial Sequence
  Synthetic construct
RRVWRWVRRVWRWVRR

SEQ ID NO: 15 - protein, 18 aa, Artificial Sequence
  Synthetic construct
RRVWRWVRRVWRRVRRWV

SEQ ID NO: 16 - protein, 20 aa, Artificial Sequence
  Synthetic construct
RRVWRWVRRVWRWVRRVVRR

SEQ ID NO: 17 - protein, 22 aa, Artificial Sequence
  Synthetic construct
RRVWRWVRRVWRWVRRVVRRVR

SEQ ID NO: 18 - protein, 22 aa, Artificial Sequence
  Synthetic construct
RRVRRWVRRVWRWVRRVWRRVR

SEQ ID NO: 19 - protein, 24 aa, Artificial Sequence
  Synthetic construct
RRVWRWVRRVWRWVRRVVRRVRRV

SEQ ID NO: 20 - protein, 24 aa, Artificial Sequence
  Synthetic construct
RRVRRWVRRVWRWVRRVWRRVRRV

SEQ ID NO: 21 - protein, 10 aa, Artificial Sequence
  Synthetic construct
RRWWRRWWRW

SEQ ID NO: 22 - protein, 12 aa, Artificial Sequence
  Synthetic construct
RRWWRWVRRWWR

SEQ ID NO: 23 - protein, 14 aa, Artificial Sequence
  Synthetic construct
RRWWRWVRRWWRRV

SEQ ID NO: 24 - protein, 16 aa, Artificial Sequence
  Synthetic construct
RRVWRWWRRVWRWVRR

SEQ ID NO: 25 - protein, 18 aa, Artificial Sequence
  Synthetic construct
RRVWRWVRRVWRRVRRWW

SEQ ID NO: 26 - protein, 8 aa, Artificial Sequence
  Synthetic construct
WRRRWRRW

SEQ ID NO: 27 - protein, 14 aa, Artificial Sequence
  Synthetic construct
VWRRVRRWVRRWRR

SEQ ID NO: 28 - protein, 16 aa, Artificial Sequence
  Synthetic construct
VWRRWRRWVRRVRRRV

SEQ ID NO: 29 - protein, 18 aa, Artificial Sequence
  Synthetic construct
VWRRVRRVVRRWRRWVRR

SEQ ID NO: 30 - protein, 20 aa, Artificial Sequence
  Synthetic construct
RVWRRVRRVVRRWRRWVRRV

SEQ ID NO: 31 - protein, 22 aa, Artificial Sequence
  Synthetic construct
RRVWRRVRRVVRRWRRWVRRVV

SEQ ID NO: 32 - protein, 24 aa, Artificial Sequence
  Synthetic construct
RRVVRRWRRVVRRVWRWVRRVVRR

SEQ ID NO: 33 - protein, 8 aa, Artificial Sequence
  Synthetic construct
RRWWRRWW

SEQ ID NO: 34 - protein, 10 aa, Artificial Sequence
  Synthetic construct
WWRRWRRRWR

SEQ ID NO: 35 - protein, 16 aa, Artificial Sequence
  Synthetic construct
RRWWRVVRRVVRWWRR

SEQ ID NO: 36 - protein, 18 aa, Artificial Sequence
  Synthetic construct
RRWWRWVRRVVRRWRRVV

SEQ ID NO: 37 - protein, 20 aa, Artificial Sequence
  Synthetic construct
RRWWRVVRRVVRWWRRVVRR

SEQ ID NO: 38 - protein, 16 aa, Artificial Sequence
  Synthetic construct
RRWWRWVRRVVRWWRR

SEQ ID NO: 39 - protein, 16 aa, Artificial Sequence
  Synthetic construct
RRWWRWVRRVWRWVRR

SEQ ID NO: 40 - protein, 18 aa, Artificial Sequence
  Synthetic construct
RRVWRWVRRVWRRWRRWV

SEQ ID NO: 41 - protein, 18 aa, Artificial Sequence
  Synthetic construct
RRWWRWWRRVVRRWRRVV

SEQ ID NO: 42 - protein, 12 aa, Artificial Sequence
  Synthetic construct
VVRRWRRRWRRW

SEQ ID NO: 43 - protein, 14 aa, Artificial Sequence
  Synthetic construct
WVRRWRRVVRRWRR

SEQ ID NO: 44 - protein, 16 aa, Artificial Sequence
  Synthetic construct
VVRRWRRVVRRWRRRW

SEQ ID NO: 45 - protein, 18 aa, Artificial Sequence
  Synthetic construct
WVRRWRRVVRRWRRVVRR

SEQ ID NO: 46 - protein, 20 aa, Artificial Sequence
  Synthetic construct
RWVRRWRRVVRRWRRVVRRV

SEQ ID NO: 47 - protein, 22 aa, Artificial Sequence
  Synthetic construct
RRWVRRWRRVVRRWRRVVRRVV

SEQ ID NO: 48 - protein, 24 aa, Artificial Sequence
  Synthetic construct
RRVVRRWRRVVRRWVRVWRRVVRR

SEQ ID NO: 49 - protein, 12 aa, Artificial Sequence
  Synthetic construct
RRWVRVWRRWWR

SEQ ID NO: 50 - protein, 14 aa, Artificial Sequence
  Synthetic construct
RRWVRVWRRWVRRW

SEQ ID NO: 51 - protein, 14 aa, Artificial Sequence
  Synthetic construct
RRWVRVWRRWWRRV

SEQ ID NO: 52 - protein, 16 aa, Artificial Sequence
  Synthetic construct
RRWVRVWRRWVRVWRR

SEQ ID NO: 53 - protein, 18 aa, Artificial Sequence
  Synthetic construct
RRWVRVWRRWVRRWRRVV

SEQ ID NO: 54 - protein, 20 aa, Artificial Sequence
  Synthetic construct
RRWVRVWRRWVRVWRRVVRR

SEQ ID NO: 55 - protein, 22 aa, Artificial Sequence
  Synthetic construct
RRWRRVVRRWVRVWRRWVRRVR

SEQ ID NO: 56 - protein, 22 aa, Artificial Sequence
  Synthetic construct
RRWRRVVRRWVRVWRRVVRRWR

SEQ ID NO: 57 - protein, 24 aa, Artificial Sequence
  Synthetic construct
RRWRRVVRRWVRVWRRVVRRWRRV

SEQ ID NO: 58 - protein, 14 aa, Artificial Sequence
  Synthetic construct
RRWVRVWRRWWRRW

SEQ ID NO: 59 - protein, 16 aa, Artificial Sequence
  Synthetic construct
RRWVRWWRRWVRVWRR

SEQ ID NO: 60 - protein, 18 aa, Artificial Sequence
  Synthetic construct
RRWVRVWRRWVRRWRRVW

SEQ ID NO: 61 - protein, 10 aa, Artificial Sequence
  Synthetic construct
VWRRWRRRWR

SEQ ID NO: 62 - protein, 12 aa, Artificial Sequence
  Synthetic construct
WVRRWRRRVRRW

SEQ ID NO: 63 - protein, 14 aa, Artificial Sequence
  Synthetic construct
VWRRWRRVWRRVRR

SEQ ID NO: 64 - protein, 16 aa, Artificial Sequence
  Synthetic construct
VWRRVRRVWRRVRRRW

SEQ ID NO: 65 - protein, 18 aa, Artificial Sequence
  Synthetic construct
VWRRVRRVWRRVRRVWRR

SEQ ID NO: 66 - protein, 20 aa, Artificial Sequence
  Synthetic construct
RWVRRVRRWVRRVRRWVRRV

SEQ ID NO: 67 - protein, 20 aa, Artificial Sequence
  Synthetic construct
RVWRRVRRVWRRVRRVWRRV

SEQ ID NO: 68 - protein, 20 aa, Artificial Sequence
  Synthetic construct
RWVRRVRRWVRRVRRVVRRW

SEQ ID NO: 69 - protein, 22 aa, Artificial Sequence
  Synthetic construct
RRVWRRVRRVWRRVRRVVRRVW

SEQ ID NO: 70 - protein, 22 aa, Artificial Sequence
  Synthetic construct
RRWVRRVRRWVRRVRRVVRRWV

SEQ ID NO: 71 - protein, 22 aa, Artificial Sequence
  Synthetic construct
RRWVRRVRRWVRRVRRWVRRVV

SEQ ID NO: 72 - protein, 22 aa, Artificial Sequence
  Synthetic construct
RRVWRRVRRVWRRVRRVWRRVV

SEQ ID NO: 73 - protein, 10 aa, Artificial Sequence
  Synthetic construct
RRWWRRWVRW

SEQ ID NO: 74 - protein, 12 aa, Artificial Sequence
  Synthetic construct
RRWWRVWRRVWR

SEQ ID NO: 75 - protein, 12 aa, Artificial Sequence
  Synthetic construct
VWRRWRRRWRRW

SEQ ID NO: 76 - protein, 14 aa, Artificial Sequence
  Synthetic construct
RRWVRWVRRWVRRW

SEQ ID NO: 77 - protein, 16 aa, Artificial Sequence
  Synthetic construct
RRWVRWVRRWVRWVRR

SEQ ID NO: 78 - protein, 18 aa, Artificial Sequence
  Synthetic construct
RRWVRWVRRWVRRVRRWV

SEQ ID NO: 79 - protein, 20 aa, Artificial Sequence
  Synthetic construct
RRVWRVWRRVWRVVRRVWRR

SEQ ID NO: 80 - protein, 12 aa, Artificial Sequence
  Synthetic construct
RRWWRVWRRWWR

SEQ ID NO: 81 - protein, 14 aa, Artificial Sequence
  Synthetic construct
RRWVRWWRRWVRRW

SEQ ID NO: 82 - protein, 16 aa, Artificial Sequence
  Synthetic construct
RRWVRWVRRWVRWWRR

SEQ ID NO: 83 - protein, 18 aa, Artificial Sequence
  Synthetic construct
RRVWRVWRRVWRRWRRVW

SEQ ID NO: 84 - protein, 14 aa, Artificial Sequence
  Synthetic construct
VWRRVRRWWRRVRR

SEQ ID NO: 85 - protein, 16 aa, Artificial Sequence
  Synthetic construct
VWRRVRRWWRRVRRRV

SEQ ID NO: 86 - protein, 22 aa, Artificial Sequence
  Synthetic construct
RRVWRRVRRVWRRVRRWVRRVV

SEQ ID NO: 87 - protein, 24 aa, Artificial Sequence
  Synthetic construct
RRWWRRVRRVVRRVVRVVRRWVRR

(19) United States
(12) Patent Application Publication
(10) Pub. No.: US 2024/0197823 A1
(43) Pub. Date: Jun. 20, 2024

(54) CATIONIC ANTIMICROBIAL PEPTIDES AND USES THEREOF

(71) Applicant: UNIVERSITY OF PITTSBURGH-OF THE COMMONWEALTH SYSTEM OF HIGHER EDUCATION, Pittsburgh, PA (US)

(72) Inventor: Berthony DESLOUCHES, Pittsburgh, PA (US)

(21) Appl. No.: 18/276,978

(22) PCT Filed: Feb. 14, 2022
(86) PCT No.: PCT/US2022/016299

§ 371 Date: Aug. 11, 2023

Related U.S. Application Data
(60) Provisional application No. 63/148,860, filed on Feb. 12, 2021.

(51) Int. Cl. A61K 38/17; A61P 31/04

(52) U.S. Cl. CPC A61K 38/1729; A61P 31/04

(57) ABSTRACT

Provided herein are compositions comprising an antimicrobial cationic amphipathic polypeptide (PAX) and methods of using the same for treatment of a microbial infection

### CROSS-REFERENCE TO RELATED APPLICATIONS

This application claims the benefit of U.S. Provisional Application No. 63/148,860, filed Feb. 12, 2021, which is expressly incorporated herein by reference in its entirety.

### STATEMENT REGARDING FEDERALLY SPONSORED RESEARCH OR DEVELOPMENT

This invention was made with government support under grant number GM125917 awarded by the National Institutes of Health. The government has certain rights in the invention.

### FIELD

The present disclosure relates to the field of treating microbial infections and antimicrobial peptides.

### BACKGROUND

The membrane-perturbing mechanism of many antimicrobial peptides makes an appealing case for their development as an effective therapeutic source against infections associated with multidrug-resistant (MDR) bacteria [Deslouches B et al., 2020; Casciaro B et al., 2017; Magana M et al., 2020]. The magnitude of the crisis constituted by bacterial resistance to traditional antibiotics has elicited an immense effort to investigate such a diverse class of agents [Mwangi J et al., 2019; de Breij A et al., 2018]. Since their discoveries as the first line of the innate defense system against infectious pathogens over four decades ago, AMPs have been the subject of a vast literature, revealing thousands of natural AMPs across most life forms and extensive data on structure-function correlations [Zouhir A et al., 2017; Bishop B M et al., 2017]. Despite all these efforts, most classically synthesized AMPs, which are made with conventional amino acids, have failed some of the rigorous tests in advanced phases of clinical development. In addition, clinical development is typically directed toward local delivery to the sites of infection [Mwangi J et al., 2019; de Breij A et al., 2018; Di Y P et al., 2020; Jiang S et al., 2019; Mandell J B et al., 2017; Mourtada R et al., 2019]. The lack of clinical evidence for systemic use after decades of research often leaves the AMP field with a sense that the expectations from the promising data on AMPs can never come to fruition. One apparent reason is that the AMP field only advances incrementally. In addition, while many discoveries seem promising, these advances often lead to scattered information that is sometimes repetitive without resulting in specific and definitive structure-function correlations, which can reduce the need for trial and error in AMP design and structural optimization. Further, a remarkable challenge in AMP design is to increase antibacterial potency without increasing risk of host toxicity.

Accordingly, what is needed are new antimicrobial peptides for treating or preventing infection that have increased antibacterial potency and/or decreased host toxicity. The compositions and methods disclosed herein address these and other needs.

### SUMMARY

The antimicrobial cationic amphipathic polypeptides and methods disclosed herein address the certain unmet needs in treating or preventing infection. In some aspects, disclosed herein is a composition comprising an antimicrobial cationic amphipathic polypeptide (PAX), wherein the PAX comprises at least three tryptophan residues. In some embodiments, the PAX further comprises at least four arginine residues. In some embodiments, the PAX further comprises one or more valine residues. In some embodiments, the PAX is 20 to 24 amino acids in length and comprises three tryptophan residues in the same positions as a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 69, SEQ ID NO: 70, SEQ ID NO: 71, SEQ ID NO: 72, SEQ ID NO: 86 and SEQ ID NO: 87 and has at least 85% or at least 90% sequence identity to that same sequence. In some embodiments, the PAX is 16 amino acids in length and comprises four tryptophan residues in the same positions as a sequence selected from the group consisting of a sequence selected from the group consisting of SEQ ID NO: 14, SEQ ID NO: 35, SEQ ID NO: 52 and SEQ ID NO: 77 and has at least 85% or at least 90% sequence identity to that same sequence. In some embodiments, the PAX is 14 to 18 amino acids in length and comprises five tryptophan residues in the same positions as a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83 and has at least 85% or at least 90% sequence identity to that same sequence. In some embodiments, the PAX comprises a sequence of one of SEQ ID NOs: 1 to 87.

Also disclosed herein is a method of treating or preventing a microbial infection (e.g., bacterial infection) in a subject in need thereof comprising administering to the subject a therapeutically effective amount of the PAX of any preceding aspect. The compositions and methods disclosed herein result in surprisingly effective treatment of a microbial infection. In examples, the compositions and methods disclosed herein result in surprisingly effective treatment of an infection of a drug-resistant bacteria (e.g., a multiple-drug resistant bacteria).

### DESCRIPTION OF DRAWINGS

FIGS. 1A-1C show determinants of selectivity of engineered W-rich cationic amphipathic peptides (AMPs), peptide library I. (FIG. 1A) Helical wheels of 18-residue AMPs with W residues on both sides of the hydrophobic-hydrophilic interface representing W-positional group I (library 1) from which primary sequences are deduced and shown in Table 1 (excluding minor W-positional variants). Antibacterial (mean MIC in μM, FIGS. 1B and 1C) and erythrolytic effects of AMPs at the maximum test concentration of 32 μM (FIG. 1C, bottom panel) are shown as a function of length in the context of W content. The MIC is the minimum drug concentration that results in no detectable bacterial growth in MHB2; black, 3W; green, 4W; red, 5W limited to 18 residues in length. Underlined numbers (black) indicate MIC<10 μM for GNB or GPB and RBC lysis <25%. The results are data points from 2-3 independent experimental trials.

FIGS. 2A-2C show determinants of selectivity of engineered W-rich cationic amphipathic peptides (AMPs) with Trp positional group II (Library 2). (FIG. 2A) Helical wheels of 18-residue AMPs with W residues asymmetrically aggregated at the hydrophilic-hydrophobic interface, which represents W positional group II and corresponds to primary sequences shown in Table 2 (excluding the W-positional variants). The antibacterial (mean MIC in μM, FIGS. 2B and 2C) and erythrolytic effects of AMPs at the maximum test concentration of 32 μM (FIG. 2C) are shown as a function of length in the context of Trp content. (Black, 3W; green, 4W; red, 5W limited to 18 residues in length). Underlined numbers (black) indicate MIC<10 μM for GNB or GPB and RBC lysis <25%. The results are data points from 2-3 independent experimental trials.

FIGS. 3A-3C show determinants of selectivity of engineered W-rich cationic amphipathic peptides (AMPs) with Trp positional group III (Library 3). (FIG. 3A) Helical wheels of 18-residue AMPs with W residues placed as far as possible from the hydrophilic domain, which represents W positional group II and corresponds to primary sequences shown in Table 3 (excluding minor W-positional variants). The antibacterial (mean MIC in μM, FIGS. 3B and 3C) and erythrolytic effects of AMPs at the maximum test concentration of 32 μM (FIG. 3C) are shown as a function of length in the context of Trp content. (Black, 3W; green, 4W; red, 5W limited to 18 residues in length). Underlined numbers (black) indicate MIC<10 μM for GNB or GPB and RBC lysis <25%. The results are data points from 2-3 independent experimental trials.

FIGS. 4A-4C show determinants of selectivity of engineered W-rich cationic amphipathic peptides (AMPs) with Trp positional group IV (Library 4). (FIG. 4A) Helical wheels of 18-residue AMPs with W residues staggered in the hydrophobic domain, which represents W positional group II and corresponds to primary sequences shown in Table 4 (excluding the W-positional variants). The antibacterial (mean MIC in μM, FIGS. 4B and 4C) and erythrolytic effects of AMPs at the maximum test concentration of 32 μM (FIG. 4C) are shown as a function of length in the context of Trp content. (Black, 3W; green, 4W; red, 5W limited to 18 residues in length). Underlined numbers (black) indicate MIC<10 μM for GNB or GPB and RBC lysis <25%. The results are data points from 2-3 independent experimental trials.

FIGS. 5A-5B show antibacterial activity and toxicity of selected engineered W-rich peptide antibiotics (PAX). Selected PAX were examined for minimum inhibitory concentrations (MIC, FIG. 5A) against GNB and GPB MDR isolates from the CDC shown in S4 Table. Toxicity against freshly isolated human white blood cells (FIG. 5B, WBC) was determined by live-dead stain incorporation detected by flow cytometry. Data are representative of 2-3 experimental trials with all data points included in FIG. 5A, whereas one of two representative independent experimental trials is shown in FIG. 5B. Green bar below E35 (X axis, FIG. 5B) indicates a single PAX selected for more advanced studies; Tobi, tobramycin; Ceft, ceftazidime; Oxa, oxacillin; Ttrp, tritrpticin; Ctrl-0, no agent added; SAAP, LL37-derived SAAP-148.

FIGS. 6A-6F show selective killing mechanism. PAX E35 (hPAXII-17 of 16 residues shown in green, FIG. 1 and Table 1) was first tested for bactericidal activity and killing kinetics against P. aeruginosa strain PA239 in fetal bovine serum containing medium (FIGS. 6A and 6B), with tobramycin as control. E35 interaction with membrane mimics was characterized by circular dichroism (FIGS. 6C and 6D). Finally, Membrane perturbation was examined by propidium Iodide incorporation detected by flow cytometry using PA239 treated with E35 (FIG. 6E) and tobramycin (FIG. 6F). Data are representative of two independent experimental trials. Membrane mimics: G(+), gram positive; [Beringer P M et al., 2016], gram negative; Euk, eukaryotic.

FIGS. 7A-7C show efficacy of hPAXII-17 (E35) against P. aeruginosa PA239. CD-1 mice (N=13) were infected IP (intraperitoneally) with PA239 (˜3×10⁷ CFU) and randomly selected to receive PBS or PAX E35 at 5 mg/kg). The animals were protected from the infection, except for 1 of the 13 treated mice (FIG. 7A). After 4 h post-treatment, bacterial burden in the blood was reduced by >10-fold (FIG. 7B), reflective of the marked reduction of bacterial load in the lungs compared to the mock-treated mice (FIG. 7C), consistent with the high rate of survival (FIG. 7A). Statistical significance was determined by the Log-rank (Mantel-Cox) and Gehan-Breslow-Wilcoxon tests (FIG. 7A) as well as unpaired t test with two-tailed P values (FIGS. 7B and 7C).

FIGS. 8A-8B show in vivo efficacy of E35 against K. pneumoniae (KP125). Mice were immunosuppressed with IP injection of cyclophosphamide, 150 mpk on induction and 120 mpk daily×4 days. Infection was induced on day 6 (10⁷ CFU IP). Daily treatment with 4 mpk IV for 2 days was initiated ˜2-2.5 h post-exposure to the bacterial inoculum. Bacterial burden, 48 h post-infection (˜20 h after the second dose of treatment), was determined by necropsies, and enumeration on agar plates; (FIG. 8A) lungs and (FIG. 8B) blood; statistical analysis by Kolmogorov-Smirnov test; P=0.0079 for both lungs and blood.

FIG. 9 shows activities of antimicrobial agents used as controls in screening corresponding to FIGS. 1 and 2, 9 and 10. Different controls were used against GNB and GPB: Indl, indolicidin; Trp, tritrpticin; SAAP, SAAP-148; K6L9. Used against only GNB were Tobi (tobramycin), Mero (meropenem) and Ceft (ceftazidime). Finally, Cefa (cefazolin) was used only against GPB. Shown at the bottom is the percent red blood cell lysis at only the maximum test concentration of 32 μM for the control AMPs in dose-response assays. Broken line shows mean MIC at or below 10 μM for clarity. Data plotted in B and C represent individual MICs from 2 to 3 independent experimental trials for each bacterial strain. Please refer to Table 5 for individual MICs.

FIGS. 10A-10C show mean residue ellipticity (MRE) of E2-0035 with increasing lipid:peptide molar ratios for three membrane mimics. FIG. 10A. gram-negative inner bacterial membrane, FIG. 10B. Gram-positive cellular membrane, FIG. 10C. Eukaryotic membrane containing 33 mole % cholesterol. Tables on right of each graph quantitate the four secondary structural motifs at each molar ratio. Adjusted R2 shows the goodness of the fit.

### DETAILED DESCRIPTION

Disclosed herein are cationic amphipathic peptide antibiotics (PAXs), wherein the PAX comprises an equal number of cationic and hydrophobic amino acids. In some embodiments, the PAX is 8, 10, 12, 14, 16, 18, 20, 22, or 24 amino acids in length. These cationic PAXs have been shown to be surprisingly effective at treating and/or preventing microbial infections, including, for example, infections of drug-resistant bacteria. Accordingly, disclosed herein are methods of treating and/or preventing microbial infections comprising administering to a subject indeed thereof a therapeutically effective amount of the cationic amphipathic PAXs disclosed herein. As one example, the cationic amphipathic PAXs show effective anti-microbial effects without substantial toxicity to host cells.

Terms used throughout this application are to be construed with ordinary and typical meaning to those of ordinary skill in the art. However, Applicants desire that the following terms be given the particular definition as provided below.

### Terminology

As used in the specification and claims, the singular form “a,” “an,” and “the” include plural references unless the context clearly dictates otherwise. For example, the term “a cell” includes a plurality of cells, including mixtures thereof.

The term “about” as used herein when referring to a measurable value such as an amount, a percentage, and the like, is meant to encompass variations of ±20%, ±10%, ±5%, or ±1% from the measurable value.

“Administration” to a subject includes any route of introducing or delivering to a subject an agent (e.g. a composition disclosed herein). Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the term “amphipathic” refers to a molecule, especially a polypeptide, having both hydrophilic and hydrophobic parts. The term “amphiphilic polypeptide” refers to a polypeptide that possesses both hydrophilic and hydrophobic properties.

“Antimicrobial” refers to the ability to kill or inhibit the growth of a microbe.

The term “biocompatible” generally refers to a material and any metabolites or degradation products thereof that are generally non-toxic to the recipient and do not cause significant adverse effects to the subject.

“Cationic” as is used to refer to any composition (e.g., a polypeptide) having a net positive charge. In some embodiments, a cationic polypeptide has net positive charge. In some embodiments, a cationic polypeptide comprises a surface-active cation.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

“Composition” refers to any agent that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a bacterial infection). The terms also encompass pharmaceutically acceptable, pharmacologically active derivatives of beneficial agents specifically mentioned herein, including, but not limited to, a vector, polynucleotide, cells, salts, esters, amides, proagents, active metabolites, isomers, fragments, analogs, and the like. When the term “composition” is used, then, or when a particular composition is specifically identified, it is to be understood that the term includes the composition per se as well as pharmaceutically acceptable, pharmacologically active vector, polynucleotide, salts, esters, amides, proagents, conjugates, active metabolites, isomers, fragments, analogs, etc. In some aspects, the composition disclosed herein comprises a cationic anti-microbial polypeptide.

When referring to a microbe, the term “drug resistant” or “resistant” refers to a microbe, or a population of the microbe, that is less susceptible to damage or killing by a drug. Drug resistance occurs due to a change in the microbe population over time that makes the microbe population less susceptible to the drug. In some embodiments, there is an about 100%, about 90%, about 80%, about 70%, about 60%, about 50% or about 40% reduction in the effectiveness of a drug on a drug resistant microbe or microbe population as compared to a control.

By the term “effective amount” of a therapeutic agent is meant a nontoxic but sufficient amount of a beneficial agent to provide the desired effect. The amount of beneficial agent that is “effective” will vary from subject to subject, depending on the age and general condition of the subject, the particular beneficial agent or agents, and the like. Thus, it is not always possible to specify an exact “effective amount.” However, an appropriate “effective” amount in any subject case may be determined by one of ordinary skill in the art using routine experimentation. Also, as used herein, and unless specifically stated otherwise, an “effective amount” of a beneficial can also refer to an amount covering both therapeutically effective amounts and prophylactically effective amounts. An “effective amount” of a drug necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc. In any case, the fragment must possess a bioactive property, such as regulating the transcription of the target gene.

The terms “helix” or “helical polypeptide” used herein refers any protein or peptide secondary structure that forms a spiral.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%,94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity over the full-length of the compared sequences. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

The term “MIC” refers to “minimal inhibitory concentration” and is defined as the lowest concentration of a peptide that prevents visible growth of a microbe such as a bacterium or bacteria.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The terms “peptide,” “protein,” and “polypeptide” are used interchangeably to refer to a natural or synthetic molecule comprising two or more amino acids linked by the carboxyl group of one amino acid to the alpha amino group of another.

The term “polypeptide” refers to a compound made up of a single chain of D- or L-amino acids or a mixture of D- and L-amino acids joined by peptide bonds.

The term “reduced”, “reduce”, “reduction”, or “decrease” as used herein generally means a decrease by a statistically significant amount. However, for avoidance of doubt, “reduced” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e., absent level as compared to a reference sample), or any decrease between 10-100% as compared to a reference level.

The term “subject” is defined herein to include animals such as mammals, including, but not limited to, primates (e.g., humans), cows, sheep, goats, horses, dogs, cats, rabbits, rats, mice, and the like. In some embodiments, the subject is a human.

The terms “treat,” “treating,” “treatment,” and grammatical variations thereof as used herein, include partially or completely alleviating, mitigating, or reducing the intensity of one or more attendant symptoms of a disorder or condition and/or alleviating, mitigating, or impeding one or more causes of a disorder or condition. In some instances, the terms “treat”, “treating”, “treatment” and grammatical variations thereof, include reducing a bacterial infection in a subject, reducing the amount of a bacterial load in a subject or a tissue and/or reducing the level of inflammation in a subject or a tissue as compared with prior to treatment of the subject or as compared with the incidence of such symptom in a general or study population.

“Therapeutically effective amount” or “therapeutically effective dose” of a composition (e.g., a composition comprising an agent) refers to an amount that is effective to achieve a desired therapeutic result. In some embodiments, a desired therapeutic result is the control of a bacterial infection, a reduction of a bacterial infection, or the mitigation of a symptom of a bacterial infection. In some embodiments, a desired therapeutic result is the reduction of a bacterial load in a subject. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

### Compositions

Natural antimicrobial peptides (AMPs) are a class of therapeutics with potential efficacy against drug-resistant related infections and cancer. However, several challenges have hampered their development for clinical applications, notably lack of in vivo efficacy, toxicity to mammalian cells, and direct inhibition of activity in certain biological matrices. What is needed are new antimicrobial peptides for treating a microbial infection that have an increased antimicrobial potency and/or decreased host toxicity. In some embodiments, the antimicrobial polypeptide (PAX) is antibacterial and has an increased antibacterial potency and/or decreased host toxicity as compared to a control.

Disclosed herein are compositions comprising an antimicrobial polypeptide (PAX), wherein the polypeptide is cationic and amphipathic. In some embodiments, the PAX comprises an equal number of cationic amino acids and hydrophobic amino acids.

The term “cationic amino acid” refers to the amino acid with cationic side chain. In some embodiments, the cationic amino acid is selected form the group consisting of the L or D form of lysine, histidine, and arginine. The term “hydrophobic amino acid” refers to the amino acid with hydrophobic side chain. the hydrophobic amino acid is selected from the group consisting of the L or D form of the following: alanine, isoleucine, leucine, tryptophan, phenylalanine, valine, praline, and glycine. “Cationic polypeptide” is used to refer to a polypeptide having a net positive charge. In some embodiments, the cationic PAX disclosed herein comprises an equal number of cationic and hydrophobic amino acids, wherein the cationic amino acids are arginine. In some embodiments, the hydrophobic amino acids are selected from valine and tryptophan.

Amino acid codes known to those skilled in the art and provided in the table below will be used throughout this disclosure.

Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

In some embodiments, the PAX is between 8 and 50 amino acids. In some embodiments, the PAX is about 10, 20, 30, 40 or 50 amino acids in length. In some embodiments, the PAX disclosed herein is at least 8, 10, 12, 14, 16, 18, 20, 22, or at least 24 amino acids in length. In some embodiments, the PAX is 8, 10, 12, 14, 16, 18, 20, 22, 24, 26, 28 or 30 amino acids in length. In some embodiments, the PAX is 8, 10, 12, 14, 16, 18, 20, 22, or 24 amino acids in length. In some embodiments, the PAX is 12, 14 or 16 amino acids in length. In some embodiments, the PAX is 14 amino acids in length.

In some embodiments, the PAX comprises a sequence of one of SEQ ID NOs: 1-87. In some embodiments, the PAX has an amino acid sequence of one of SEQ ID NOs: 1-87. In some embodiments, the PAX comprises a sequence having at least 95%, 90%, 85% or 80% sequence identity with one of SEQ ID NOs: 1-87.

In some embodiments, the PAX comprises a sequence shown in Table 9. In some embodiments, the PAX has an amino acid sequence shown in Table 9.

In some embodiments, the PAX is 20 to 24 amino acids in length and comprises a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 86 and SEQ ID NO: 87. In some embodiments, the PAX is 20 to 24 amino acids in length and comprises three tryptophan residues in the same positions as a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 86 and SEQ ID NO: 87 and has at least 85% or at least 90% sequence identity to that same sequence. In some embodiments, the PAX is 20 to 24 amino acids in length and comprises a sequence selected from the group consisting of SEQ ID NO: 7, SEQ ID NO: 8, SEQ ID NO: 31, SEQ ID NO: 48, SEQ ID NO: 66, SEQ ID NO: 67, SEQ ID NO: 68, SEQ ID NO: 69, SEQ ID NO: 72 and SEQ ID NO: 86 and has at least 85% or at least 90% sequence identity to that same sequence.

In some embodiments, the PAX comprises SEQ ID NO: 87.

In some embodiments, the PAX is 16 amino acids in length and comprises a sequence selected from the group consisting of SEQ ID NO: 14, SEQ ID NO: 35, SEQ ID NO: 52 and SEQ ID NO: 77. In some embodiments, the PAX is 16 amino acids in length and comprises four tryptophan residues in the same positions as a sequence selected from the group consisting of a sequence selected from the group consisting of SEQ ID NO: 14, SEQ ID NO: 35, SEQ ID NO: 52 and SEQ ID NO: 77 and has at least 85% or at least 90% sequence identity to that same sequence.

In some embodiments, the PAX comprises SEQ ID NO: 14.

In some embodiments, the PAX is 14 to 18 amino acids in length and comprises a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83. In some embodiments, the PAX is 14 to 18 amino acids in length and comprises five tryptophan residues in the same positions as a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83 and has at least 85% or at least 90% sequence identity to that same sequence.

In some embodiments, the PAX comprises a sequence selected from the group consisting of SEQ ID NO: 48, SEQ ID NO: 8, SEQ ID NO: 31, SEQ ID NO: 7, SEQ ID NO: 14, SEQ ID NO: 66, SEQ ID NO: 67, SEQ ID NO: 68, SEQ ID NO: 69, SEQ ID NO: 86, SEQ ID NO: 72, and SEQ ID NO: 77.

In some embodiments, the PAX comprises a sequence selected from the group consisting of SEQ ID NO: 7, SEQ ID NO: 8, SEQ ID NO: 9, SEQ ID NO: 14, SEQ ID NO: 23, SEQ ID NO: 24, SEQ ID NO: 25 and SEQ ID NO: 86.

In some embodiments, the PAX comprises a sequence selected from the group consisting of SEQ ID NO: 30, SEQ ID NO: 31, SEQ ID NO: 32, SEQ ID NO: 35, SEQ ID NO: 38, SEQ ID NO: 39, SEQ ID NO: 40 and SEQ ID NO: 41.

In some embodiments, the PAX comprises a sequence selected from the group consisting of SEQ ID NO: 46, SEQ ID NO: 47, SEQ ID NO: 48, SEQ ID NO: 52, SEQ ID NO: 58, SEQ ID NO: 59 and SEQ ID NO: 60.

In some embodiments, the PAX comprises a sequence selected from the group consisting of SEQ ID NO: 69, SEQ ID NO: 70, SEQ ID NO: 71, SEQ ID NO: 72, SEQ ID NO: 77, SEQ ID NO: 81, SEQ ID NO: 82 and SEQ ID NO: 83.

In some embodiments, the PAX is between 8 and 24 amino acids in length.

In some embodiments, the PAX comprises a sequence of one of SEQ ID NOs: 26 and 28. In some embodiments, the PAX comprises a sequence of one of SEQ ID NOs: 26 and 28 and is between 8 and 24 amino acids in length.

In some embodiments, the PAX comprises a sequence of one of SEQ ID NOs: 1, 10, 21, 34, 61, and 73. In some embodiments, the PAX comprises a sequence of one of SEQ ID NOs: 1, 10, 21, 34, 61, and 73 and is between 10 and 24 amino acids in length.

In some embodiments, the PAX comprises a sequence of one of SEQ ID NOs: 2, 11, 12, 22, 42, 49, 62, 74, 75, and 80. In some embodiments, the PAX comprises a sequence of one of SEQ ID NOs: 2, 11, 12, 22, 42, 49, 62, 74, 75, and 80 and is between 12 and 24 amino acids in length.

In some embodiments, the PAX comprises a sequence of one of SEQ ID NOs: 3, 13, 23, 27, 43, 50, 51, 58, 63, 76, 81, and 84. In some embodiments, the PAX comprises a sequence of one of SEQ ID NOs: 3, 13, 23, 27, 43, 50, 51, 58, 63, 76, 81, and 84 and is between 14 and 24 amino acids in length. In some embodiments, the PAX has the polynucleotide sequence of SEQ ID NO: 14.

In some embodiments, the PAX comprises a sequence of one of SEQ ID NOs: 4, 14, 24, 28, 35, 38, 39, 44, 52, 59, 64, 77, 82, and 85. In some embodiments, the PAX comprises a sequence of one of SEQ ID NOs: 4, 14, 24, 28, 35, 38, 39, 44, 52, 59, 64, 77, 82, and 85 and is between 16 and 24 amino acids in length.

In some embodiments, the PAX comprises a sequence of one of SEQ ID NOs: 5, 6, 15, 25, 29, 36, 40, 41, 45, 53, 60, 65, 78, and 83. In some embodiments, the PAX comprises a sequence of one of SEQ ID NOs: 5, 6, 15, 25, 29, 36, 40, 41, 45, 53, 60, 65, 78, and 83 and is between 18 and 24 amino acids in length.

In some embodiments, the PAX comprises a sequence of one of SEQ ID NOs: 7, 16, 30, 37, 46, 54, 66, 67, 68, and 79. In some embodiments, the PAX comprises a sequence of one of SEQ ID NOs: 7, 16, 30, 37, 46, 54, 66, 67, 68, and 79 and is between 20 and 24 amino acids in length.

In some embodiments, the PAX comprises a sequence of one of SEQ ID NOs: 8, 17, 18, 31, 47, 55, 56, 69, 70, 71, 72, and 86. In some embodiments, the PAX comprises a sequence of one of SEQ ID NOs: 8, 17, 18, 31, 47, 55, 56, 69, 70, 71, 72, and 86 is between 22 and 24 amino acids in length.

In some embodiments, the PAX comprises a sequence of one of SEQ ID NOs: 9, 19, 20, 32, 48, and 57. In some embodiments, the PAX comprises a sequence of one of SEQ ID NOs: 9, 19, 20, 32, 48, and 57 and is 24 amino acids in length.

In some embodiments, the PAX may be in a pharmaceutical composition further comprising a pharmaceutically acceptable carrier.

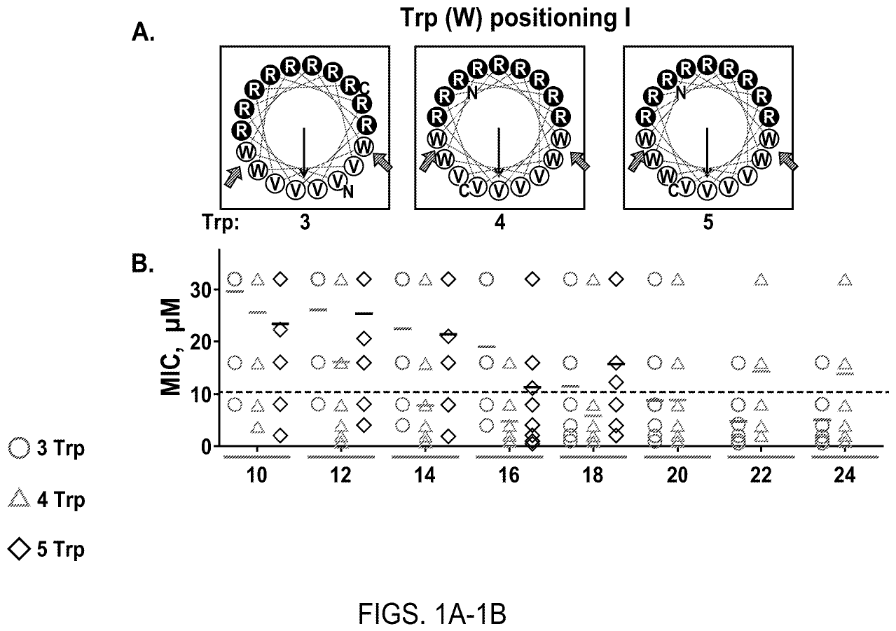

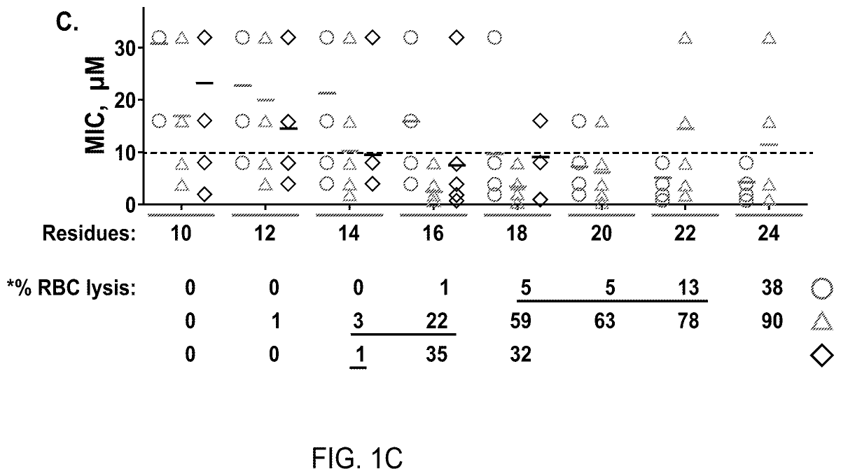

In certain aspects, the cationic amphipathic PAXs disclosed herein can form a helical structure. FIGS. 1A, 2A, 3A, and 4A herein illustrate some representative helical structures of some of the PAXs, wherein one face of the helical structure is hydrophobic and the opposite face of the helical structure is hydrophilic. Accordingly, included herein are cationic amphipathic PAXs having a helical structure wherein one face of the helical structure is hydrophobic and the opposite face of the helical structure is hydrophilic.

In some embodiments, the PAX disclosed herein possesses antimicrobial activity against a variety of microbes including, for example, bacteria, viruses, protozoa, and fungi. Notably, the PAX shows effective antimicrobial effects without substantial toxicity to host cells. In certain aspects, the microbe is a bacterium. In some embodiments, the PAX has antibacterial activity, wherein the bacterium is Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species.

In certain aspects, PAX has antibacterial activity and the bacterium is selected from the group consisting of Mycobacterium tuberculosis, Mycobacterium bovis, Mycobacterium bovis strain BCG, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, Legionella pneumophila, Bacillus anthracis, Acetinobacter baumanii, Salmonella typhi, Salmonella enterica, Shigella boydii, Shigella dysenteriae, Shigella sonnei, Shigella flexneri, Yersinia pestis, Pasteurella haemolytica, Pasteurella multocida, Actinobacillus pleuropneumoniae, Listeria monocytogenes, Listeria ivanovii, Brucella abortus, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, Haemophilus influenzae, Haemophilus ducreyi, Clostridium tetani, and Yersinia enterolitica.

In certain aspects, the PAX possess antibacterial activity against one or more of Enterococcus faecium, Staphylococcus aureus, Klebsiella pneumoniae, Acinetobacter baumannii, Pseudomonas aeruginosa, and an Enterobacter spp. Accordingly, in some embodiments, the PAX is therapeutically effective against one or more bacteria selected from the group consisting of Enterococcus faecium, Staphylococcus aureus, Klebsiella pneumoniae, Acinetobacter baumannii, Pseudomonas aeruginosa, Escherichia coli, and an Enterobacter spp. In some embodiments, the PAX is therapeutically effective against one or more bacteria selected from Escherichia coli, Pseudomonas aeruginosa, and Klebsiella pneumoniae.

Patients with infections caused by drug-resistant bacteria are at increased risk of worse clinical outcomes and death and consume more health-care resources than patients infected with non-resistant strains of the same bacteria. In some embodiments, the PAXs disclosed herein possess activity against drug-resistant bacteria.

In some embodiments, the PAXs disclosed herein possess activity against drug-resistant bacteria selected from the group consisting of Acinetobacter, Candida auris, Clostridioides difficile, Enterobacteriaceae, Neisseria gonorrhoeae, Campylobacter, ESBL-producing Enterobacteriaceae, Vancomycin-resistant Enterococcus (VRE), Pseudomonas aeruginosa, nontyphoidal Salmonella, Salmonella serotype Typhi, Shigella, Methicillin-resistant Staphylococcus aureus (S. aureus) (MRSA), Streptococcus pneumoniae, Tuberculosis, resistant Group A Streptococcus, resistant Group B Streptococcus, Mycoplasma genitalium, and Bordetella pertussis. In some embodiments, the PAXs disclosed herein possess activity against drug-resistant bacteria selected from the group consisting of Acinetobacter baumannii, Escherichia coli, Enterobacter cloacae, Klebsiella pneumoniae, Pseudomonas aeruginosa, Staphylococcus aureus, and Enterococcus faecium.

In some embodiments the PAXs disclosed herein possess activity against drug-resistant bacteria selected from Enterococcus faecium, Staphylococcus aureus, Klebsiella pneumoniae, Acinetobacter baumannii, Pseudomonas aeruginosa, and Enterobacter spp.

### Methods

Disclosed herein are methods for treating a microbial infection comprising administering to a subject in need thereof a therapeutically effective amount of the cationic amphipathic PAXs disclosed herein. In some embodiments, the treatment of a microbial infection includes a reduction of inflammation, and/or a reduction in a biofilm. Further, in certain aspects, the therapeutically effective amount is lower than a comparative AMP due to the higher potency of the PAX. Accordingly, included herein are PAX that have a lower minimum inhibitory concentration (MIC) than a comparative AMP. In other aspects, the PAX is less toxic to the subject than a comparative AMP.

The PAX used for treatment can be any as described herein. In certain aspects the PAX comprises an equal number of cationic and hydrophobic amino acids. In some embodiments, the PAX used for treatment is between 8 and 50 amino acids. In some embodiments, the PAX is about 10, 20, 30, 40 or 50 amino acids in length. In some embodiments, the PAX is at least 8, 10, 12, 14, 16, 18, 20, 22, or at least 24 amino acids in length. In some embodiments, the PAX is 8, 10, 12, 14, 16, 18, 20, 22, 24, 26, 28 or 30 amino acids in length. In some embodiments, the PAX is 8, 10, 12, 14, 16, 18, 20, 22, or 24 amino acids in length. In some embodiments, the PAX is 12, 14 or 16 amino acids in length. In some embodiments, the PAX is 14 amino acids in length.

Also included herein are methods for treating microbial infections comprising administering to a subject in need thereof a therapeutically effective amount of a cationic amphipathic PAX of any of the aforementioned lengths and any of the herein mentioned sequences having a helical structure wherein one face of the helical structure is hydrophobic and the opposite face of the helical structure is hydrophilic.

In some embodiments, the PAX used for treatment comprises a sequence of one of SEQ ID NOs: 26 and 28. In some embodiments, the PAX used for treatment comprises a sequence of one of SEQ ID NOs: 26 and 28 and is between 8 and 24 amino acids in length. In some embodiments, the PAX used for treatment comprises a sequence shown in Table 9. In some embodiments, the PAX use for treatment has a sequence shown in Table 9.

In some embodiments, the PAX used for treatment is 20 to 24 amino acids in length and comprises a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 86 and SEQ ID NO: 87. In some embodiments, the PAX used for treatment is 20 to 24 amino acids in length and consists of three tryptophan residues in the same positions as a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 86 and SEQ ID NO: 87 and has at least 85% or at least 90% sequence identity to that same sequence.

In some embodiments, the PAX used for treatment comprises SEQ ID NO: 87.

In some embodiments, the PAX used for treatment is 16 amino acids in length and comprises a sequence selected from the group consisting of SEQ ID NO: 14, SEQ ID NO: 35, SEQ ID NO: 52 and SEQ ID NO: 77. In some embodiments, the PAX used for treatment is 16 amino acids in length and consists of four tryptophan residues in the same positions as a sequence selected from the group consisting of a sequence selected from the group consisting of SEQ ID NO: 14, SEQ ID NO: 35, SEQ ID NO: 52 and SEQ ID NO: 77 and has at least 85% or at least 90% sequence identity to that same sequence.

In some embodiments, the PAX used for treatment comprises SEQ ID NO: 14.

In some embodiments, the PAX used for treatment is 14 to 18 amino acids in length and comprises a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83. In some embodiments, the PAX used for treatment is 14 to 18 amino acids in length and consists of five tryptophan residues in the same positions as a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83 and has at least 85% or at least 90% sequence identity to that same sequence.

In some embodiments, the PAX used for treatment comprises a sequence selected from the group consisting of SEQ ID NO: 48, SEQ ID NO: 8, SEQ ID NO: 31, SEQ ID NO: 7, SEQ ID NO: 14, SEQ ID NO: 66, SEQ ID NO: 67, SEQ ID NO: 68, SEQ ID NO: 69, SEQ ID NO: 86, SEQ ID NO: 72, and SEQ ID NO: 77.

In some embodiments, the PAX used for treatment comprises a sequence selected from the group consisting of SEQ ID NO: 7, SEQ ID NO: 8, SEQ ID NO: 9, SEQ ID NO: 14, SEQ ID NO: 23, SEQ ID NO: 24, SEQ ID NO: 25 and SEQ ID NO: 86.

In some embodiments, the PAX used for treatment comprises a sequence selected from the group consisting of SEQ ID NO: 30, SEQ ID NO: 31, SEQ ID NO: 32, SEQ ID NO: 35, SEQ ID NO: 38, SEQ ID NO: 39, SEQ ID NO: 40 and SEQ ID NO: 41.

In some embodiments, the PAX used for treatment comprises a sequence selected from the group consisting of SEQ ID NO: 46, SEQ ID NO: 47, SEQ ID NO: 48, SEQ ID NO: 52, SEQ ID NO: 58, SEQ ID NO: 59 and SEQ ID NO: 60.

In some embodiments, the PAX used for treatment comprises a sequence selected from the group consisting of SEQ ID NO: 69, SEQ ID NO: 70, SEQ ID NO: 71, SEQ ID NO: 72, SEQ ID NO: 77, SEQ ID NO: 81, SEQ ID NO: 82 and SEQ ID NO: 83.

In some embodiments, the PAX used for treatment is between 8 and 24 amino acids in length.

In some embodiments, the PAX used for treatment comprises a sequence of one of SEQ ID NOs: 1, 10, 21, 34, 61, and 73. In some embodiments, the PAX used for treatment comprises a sequence of one of SEQ ID NOs: 1, 10, 21, 34, 61, and 73 and is between 10 and 24 amino acids in length.

In some embodiments, the PAX used for treatment comprises a sequence of one of SEQ ID NOs: 2, 11, 12, 22, 42, 49, 62, 74, 75, and 80. In some embodiments, the PAX used for treatment comprises a sequence of one of SEQ ID NOs: 2, 11, 12, 22, 42, 49, 62, 74, 75, and 80 and is between 12 and 24 amino acids in length.

In some embodiments, the PAX used for treatment comprises a sequence of one of SEQ ID NOs: 3, 13, 23, 27, 43, 50, 51, 58, 63, 76, 81, and 84. In some embodiments, the PAX used for treatment comprises a sequence of one of SEQ ID NOs: 3, 13, 23, 27, 43, 50, 51, 58, 63, 76, 81, and 84 and is between 14 and 24 amino acids in length. In some embodiments, the PAX used for treatment has the polynucleotide sequence of SEQ ID NO: 14.

In some embodiments, the PAX used for treatment comprises a sequence of one of SEQ ID NOs: 4, 14, 24, 28, 35, 38, 39, 44, 52, 59, 64, 77, 82, and 85. In some embodiments, the PAX used for treatment comprises a sequence of one of SEQ ID NOs: 4, 14, 24, 28, 35, 38, 39, 44, 52, 59, 64, 77, 82, and 85 and is between 16 and 24 amino acids in length.

In some embodiments, the PAX used for treatment comprises a sequence of one of SEQ ID NOs: 5, 6, 15, 25, 29, 36, 40, 41, 45, 53, 60, 65, 78, and 83. In some embodiments, the PAX used for treatment comprises a sequence of one of SEQ ID NOs: 5, 6, 15, 25, 29, 36, 40, 41, 45, 53, 60, 65, 78, and 83 and is between 18 and 24 amino acids in length.

In some embodiments, the PAX used for treatment comprises a sequence of one of SEQ ID NOs: 7, 16, 30, 37, 46, 54, 66, 67, 68, and 79. In some embodiments, the PAX used for treatment comprises a sequence of one of SEQ ID NOs: 7, 16, 30, 37, 46, 54, 66, 67, 68, and 79 and is between 20 and 24 amino acids in length.

In some embodiments, the PAX used for treatment comprises a sequence of one of SEQ ID NOs: 8, 17, 18, 31, 47, 55, 56, 69, 70, 71, 72, and 86. In some embodiments, the PAX used for treatment comprises a sequence of one of SEQ ID NOs: 8, 17, 18, 31, 47, 55, 56, 69, 70, 71, 72, and 86 is between 22 and 24 amino acids in length.

In some embodiments, the PAX used for treatment comprises a sequence of one of SEQ ID NOs: 9, 19, 20, 32, 48, and 57. In some embodiments, the PAX used for treatment comprises a sequence of one of SEQ ID NOs: 9, 19, 20, 32, 48, and 57 and is 24 amino acids in length. In some embodiments, the PAX used for treatment comprises the sequence of SEQ ID NO: 87. In some embodiments, the PAX used for treatment comprises the sequence of SEQ ID NO: 14.

In some embodiments, the PAX treats an infection by a bacterium, virus, protozoa, or fungus. In some embodiments, the methods comprise administration of a PAX for treatment of a bacterial infection. The bacteria can be Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, or other Yersinia species.

In certain aspects, the methods comprise administration of a therapeutically effective amount of a PAX for treatment of a bacterial infection wherein the bacteria is selected from the group consisting of Enterococcus faecium, Staphylococcus aureus, Klebsiella pneumoniae, Acinetobacter baumannii, Pseudomonas aeruginosa, Escherichia coli, and an Enterobacter spp. In some embodiments, the bacteria is Escherichia coli, Pseudomonas aeruginosa, or Klebsiella pneumoniae.

Patients with infections caused by drug-resistant bacteria are at increased risk of worse clinical outcomes and death and consume more health-care resources than patient infected with non-resistant strains of the same bacteria. Accordingly, in some embodiments, the PAXs disclosed herein can be used for treating infections of drug-resistant bacteria.

In some embodiments the PAXs disclosed herein can be used for treating infections by drug-resistant bacteria that are selected from the group consisting of Enterococcus faecium, Staphylococcus aureus, Klebsiella pneumoniae, Acinetobacter baumannii, Pseudomonas aeruginosa, and Enterobacter spp.

In some embodiments, the PAXs disclosed herein can be used for treating infections of one or more drug-resistant bacteria selected from the group consisting of Acinetobacter, Candida auris, Clostridioides difficile, Enterobacteriaceae, Neisseria gonorrhoeae, Campylobacter, ESBL-producing Enterobacteriaceae, Vancomycin-resistant Enterococcus (VRE), Pseudomonas aeruginosa, nontyphoidal Salmonella, Salmonella serotype Typhi, Shigella, Methicillin-resistant Staphylococcus aureus (S. aureus) (MRSA), Streptococcus pneumoniae, Tuberculosis, resistant Group A Streptococcus, resistant Group B Streptococcus, Mycoplasma genitalium, and Bordetella pertussis.

In some embodiments, the PAXs disclosed herein can be used for treating infections of one or more drug resistant bacteria selected from the group consisting of Acinetobacter baumannii, Escherichia coli, Enterobacter cloacae, Klebsiella pneumoniae, Pseudomonas aeruginosa, Staphylococcus aureus, and Enterococcus faecium. In some embodiments, the bacteria is resistant to one or more of the drugs selected from tobramycin, meropenem, ceftazidime, oxacillin, cefazolin, and colistin as described below, or their functional equivalents.

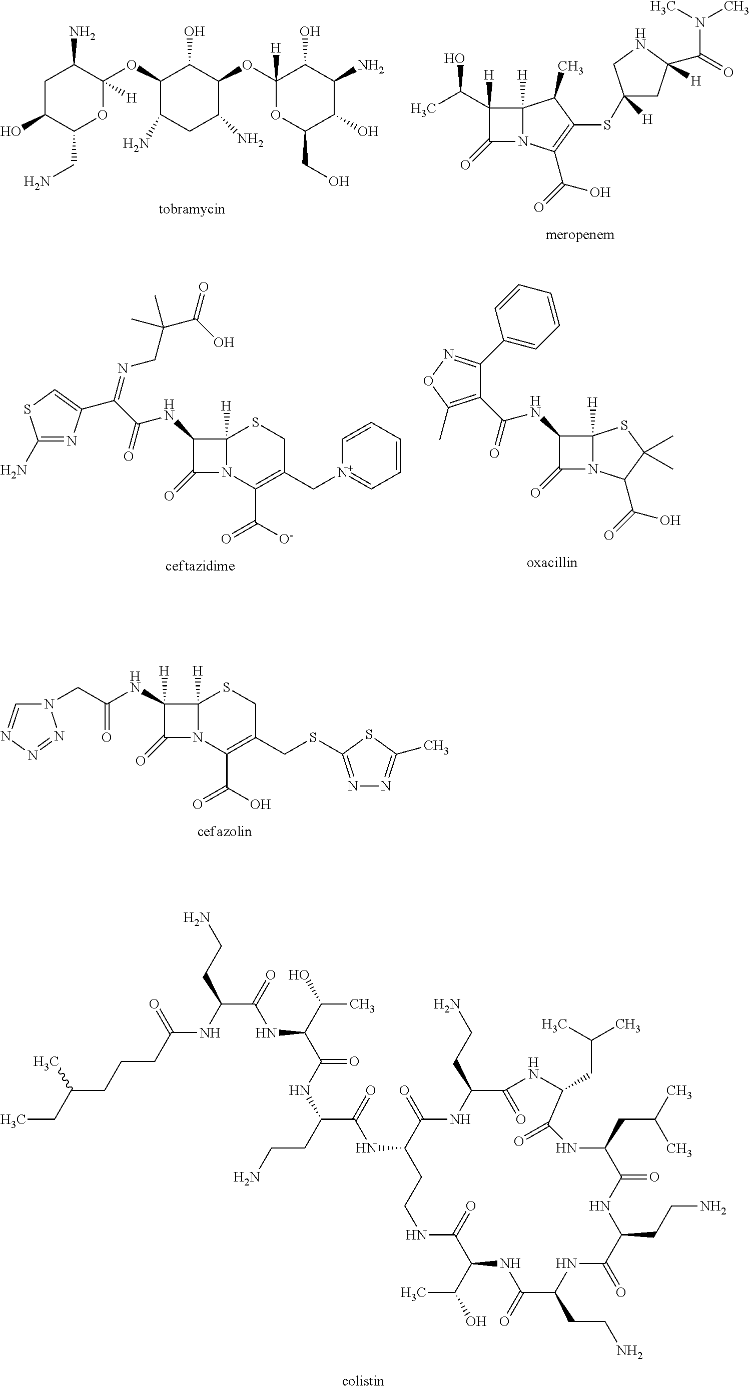

In some embodiments, the therapeutic amount of a PAX is from about 1 mg/kg body weight to about 1000 mg/kg body weight, from about 1 mg/kg to about 500 mg/kg body weight, from about 1 mg/kg to about 200 mg/kg body weight, from about 1 mg/kg to about 150 mg/kg body weight, from about 1 mg/kg to about 100 mg/kg body weight, from about 5 mg/kg to about 145 mg/kg body weight, from about 10 mg/kg to about 140 mg/kg body weight, from about 15 mg/kg to about 135 mg/kg body weight, from about 20 mg/kg to about 130 mg/kg body weight, from about 25 mg/kg to about 125 mg/kg body weight, from about 30 mg/kg to about 120 mg/kg body weight, from about 35 mg/kg to about 120 mg/kg body weight, from about 40 mg/kg to about 115 mg/kg body weight, from about 45 mg/kg to about 110 mg/kg body weight, from about 50 mg/kg to about 105 mg/kg body weight, from about 55 mg/kg to about 100 mg/kg body weight, from about 60 mg/kg to about 95 mg/kg body weight, from about 65 mg/kg to about 90 mg/kg body weight, from about 70 mg/kg to about 85 mg/kg body weight, from about 75 mg/kg to about 80 mg/kg body weight, from about 1 mg/kg to 50 mg/kg body weight, from about 1 mg/kg to 30 mg/kg body weight, from about 1 mg/kg to 20 mg/kg body weight, from about 1 mg/kg to 10 mg/kg body weight, from about 5 mg/kg to 30 mg/kg body weight, from about 5 mg/kg to 20 mg/kg body weight or a value within any of the foregoing ranges.

In some embodiments, the therapeutic amount of a PAX is from about 0.1 μM to about 32 μM, about 1 μM to about 32 μM, about 2 μM to about 30 μM, about 4 μM to about 28 μM, about 6 μM to about 26 μM, about 8 μM to about 24 μM, about 10 μM to about 22 μM, about 12 μM to about 20 μM, about 14 μM to about 18 μM, or a value within any of the forgoing ranges.

It is well known that small drug molecules cannot easily penetrate biofilm, but PAXs can penetrate and disrupt the biofilm. In some embodiments, a method of reducing a biofilm in a subject in need thereof comprises administering to the subject a therapeutic amount of an PAX. To reduce or destroy biofilm, it is believed a higher concentration of the PAX is required. In some embodiments, the therapeutic amount of an PAX is from about 1 mg/kg body weight to about 1000 mg/kg body weight, from about 1 mg/kg to about 500 mg/kg body weight, from about 1 mg/kg to about 200 mg/kg body weight, from about 1 mg/kg to about 150 mg/kg body weight, from about 1 mg/kg to about 100 mg/kg body weight, from about 5 mg/kg to about 145 mg/kg body weight, from about 10 mg/kg to about 140 mg/kg body weight, from about 15 mg/kg to about 135 mg/kg body weight, from about 20 mg/kg to about 130 mg/kg body weight, from about 25 mg/kg to about 125 mg/kg body weight, from about 30 mg/kg to about 120 mg/kg body weight, from about 35 mg/kg to about 120 mg/kg body weight, from about 40 mg/kg to about 115 mg/kg body weight, from about 45 mg/kg to about 110 mg/kg body weight, from about 50 mg/kg to about 105 mg/kg body weight, from about 55 mg/kg to about 100 mg/kg body weight, from about 60 mg/kg to about 95 mg/kg body weight, from about 65 mg/kg to about 90 mg/kg body weight, from about 70 mg/kg to about 85 mg/kg body weight, from about 75 mg/kg to about 80 mg/kg body weight, from about 1 mg/kg to 50 mg/kg body weight, from about 1 mg/kg to 30 mg/kg body weight, from about 1 mg/kg to 20 mg/kg body weight, from about 1 mg/kg to 10 mg/kg body weight, from about 5 mg/kg to 30 mg/kg body weight, from about 5 mg/kg to 20 mg/kg body weight, or a value within any of the foregoing ranges. In some embodiments, the therapeutic amount of an PAX is from about 0.1 μM to about 32 μM, about 1 μM to about 32 μM, about 2 μM to about 30 μM, about 4 μM to about 28 μM, about 6 μM to about 26 μM, about 8 μM to about 24 μM, about 10 μM to about 22 μM, about 12 μM to about 20 μM, about 14 μM to about 18 μM, or a value within any of the forgoing ranges.

In some embodiments, a method of decreasing inflammation in a subject in need thereof comprises administering to the subject a therapeutically effective amount of a PAX. The immunoregulatory response is activated once a subject is infected with an infectious microbe; when the infectious microbe is controlled by the surrounding cells secrete inflammatory cytokines, including, for example, IL-β, IL-18, IL-8, IFNγ, tumor necrosis factor (TNF), or granzyme B. This response leads to inflammation and, if left untreated or uncontrolled, can lead to tissue damage. Inflammation is controlled as the concentration of inflammatory cytokine secreted is lessened. In some embodiments, the PAX may control inflammation and the amount of inflammatory cytokine secreted without killing the infectious agent. In some embodiments, the PAX may control inflammation and the amount of inflammatory cytokine secreted and kills the infectious agent.

In certain embodiments, the pharmacokinetic (PK) properties of PAXs may be improved by conjugating molecules to the peptide. The PAXs of embodiments herein may be amidated at the C-terminus. The PAXs may be covalently bound to a protease-sensitive linker for the release of the active drug once at the site of infection. The protease-sensitive linker is selected from the group consisting of cathepsin B and polyethylene glycol (PEG) polymer. The conjugated PAX may also be packaged into liposomes or attached to nanoparticles for delivery. The PAXs may be stapled or “stitched” at 2 or more sites to enhance the pharmaceutical properties.

The disclosed methods can be performed any time following the onset of infection. In one aspect, the disclosed methods can be employed 1, 2, 3, 4, 5, 6, 7, 8, 9, 10, 15, 20, 25, 30, 35, 40, 45, 50, 55, 60, 75, 90, 105, 120 or more minutes; 3, 4, 5, 6, 7, 8, 9, 10, 11, 12, 15, 18, 24, 30, 36, 48, 60 or more hours; 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days; 4, 5, 6, 7, 8, 9, 10, 11, 12 or more months; 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1 years after the onset of infection. Dosing frequency for the composition of any preceding aspects, includes, but is not limited to, at least once every month, once every three weeks, once every two weeks, once a week, twice a week, three times a week, four times a week, five times a week, six times a week, daily, two times per day, three times per day, four times per day, five times per day, six times per day, eight times per day, nine times per day, ten times per day, eleven times per day, twelve times per day, once every 12 hours, once every 10 hours, once every 8 hours, once every 6 hours, once every 5 hours, once every 4 hours, once every 3 hours, once every 2 hours, once every hour, once every 40 min, once every 30 min, once every 20 min, or once every 10 min. Administration can also be continuous and adjusted to maintaining a level of the compound within any desired and specified range.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Rational Framework for Engineering W-Rich Peptide Antibiotics With Enhanced Selectivity Against Drug-Resistant Bacteria

Nevertheless, the lack of clinical evidence for systemic in vivo efficacy of classical AMPs should not undermine the pioneering works on AMP mechanisms and structural optimization [Martin E et al., 1995; Rozek A et al., 2000; Nguyen L T et al., 2011; Lipsky B A et al., 2008]. In particular, studies of Trp (W)-rich peptides using de novo-engineered (e.g., WLBU2) as well as natural and host-derived AMPs such as tritrpticin, indolicidin, LL37-derived SAAP-148 and ZY4 have led to the recognition of the therapeutic potential of W-based peptides in enhancing the antibacterial potency of AMPs [Mwangi J et al., 2019; de Breij A et al., 2018; Di Y P et al., 2020; Deslouches B et al., 2005]. In recent years, some of these studies have resulted in a conceptual shift from in vitro structure-function studies to proof of concept using small animal treatment models, with several AMPs now in clinical trials, including LL37 and WLBU2 (PLG0206) [Deslouches B et al., 2007; Deslouches B et al., 2005; Dijksteel G S et al., 2021; Greber K E et al., 2017]. However, most of these in vivo efficacy studies tend to focus on topical use or local delivery of the peptides to the site of administration of the bacterial inoculum [de Breij A et al., 2018; Di Y P et al., 2020].

As disclosed herein, antibacterial activity and toxicity to mammalian cells or organisms can be effectively uncoupled by the complete dissection of the cationic amphipathic motif based on well-controlled differences in Trp content and positioning, length, charge, and hydrophobic residues. Reported herein is a complete assessment of the impact of Trp on selective potency against the most common MDR bacteria known as ESKAPE pathogens. Toward this goal, multiple Trp-based libraries of cationic peptide antibiotics (PAX, designated as a distinction from natural AMPs and other engineered AMPs) were engineered (de novo), consisting of a total of 83 peptides in addition to different AMP and antibiotic controls. This study elucidates functional changes associated with minor structural differences to rationally achieve selective antibacterial properties in a way that does not appear to critically affect host toxicity. In vitro structure-function correlations resulted in rationally selected PAX candidates that displayed broad efficacy against MDR strains of ESKAPE pathogens, including those from the Center of Disease Control and Prevention (CDC). In sharp contrast to the ineffectiveness of tobramycin, one selected PAX demonstrated the ability to mitigate an otherwise lethal infection induced by an MDR strain of Pseudomonas aeruginosa in mice using a single dose given systemically.

Many structure-function studies using different host-derived AMP templates (e.g., LL37) do not necessarily result in generalized structure-function correlations because of the diversity in amino acid compositions of such templates. An explanation is that these AMP templates already include amino acids that do not necessarily inform antimicrobial functions (not typically hydrophobic or cationic), which explains AMP/PAX multifunctionality. Yet, such amino acids can influence the folding of the primary sequence in a way that partially hinders optimization specific to antimicrobial function. From an antimicrobial standpoint, these factors need to be controlled. To do so, only amino acids (typically cationic or hydrophobic) that are relevant to AMP cationic amphipathic structure were used as structural determinants of antimicrobial properties. In addition, it was important to exploit the well-studied role of Trp in partly overcoming many AMP limitations (e.g., salt, serum, host toxicity, and in vivo efficacy).

Thus, a series of PAXs were designed based on differential numbers of Trp residues (e.g., 3, 4, or 5 Trp residues, Tables 1-3 and Table 9), charge, and peptide length. Further, past studies show that Trp alone can hamper the ability to effectively uncouple antimicrobial functions from host toxicity due to its high hydrophobicity. Val was designated (instead of Phe, Leu, or Ileu because of its substantially lower hydrophobicity than that of Trp) as the amino acid of choice to complete the hydrophobic domain (with different Trp contents) as a function of length and charge. Depending on the number of Trp residues, a minimum length of 8 (4R and 4W) or 10 (5R and 5W, or 6R, 3W, and 1V) residues (Tables 1-3 and Table 9) were used. Using helical wheel projections (heliquest.fr) to deduce the primary sequences as idealized helices (FIG. 1), the length and charge of the peptides were incrementally increased by adding 1R and 1V at a time to each template until a maximum length of 24 (the 3W and 4W templates) or 18 (the 5W template) was reached. Based on Trp positional analysis, the first series (Table 2 and Table 9) was further complemented by three additional series (Tables 2-4 and Table 9), which resulted in a total number of 83 de novo-engineered PAX. As controls, Trp-containing AMPs were used such as the bovine indolicidin and tritrpticin. SAAP-148 was also used, one of the most recently engineered AMPs with remarkably broad antibacterial activity and moderate red blood cell (RBC) lysis at 16 μM. Hence, this study designed an internally controlled system using peptides that differ from each other in length by only two residues and by a charge of +1 as well as both engineered and natural AMPs that are rich in Trp content as additional controls. A well-known AMP (K6L9) with no Trp content was also included. Appropriate antibiotic controls were used depending on the test bacterial organism.

Another paramount challenge in AMP design is to increase antibacterial potency without markedly increasing the risk of host toxicity. This is partly due to varying degrees of trial and error in current approaches to AMP design. In this context, is the present study aims to show that antibacterial activity and toxicity to mammalian cells or host organisms can be effectively uncoupled by the complete dissection of the cationic amphipathic motif based on well-controlled differences in W content and positioning, length, charge, and hydrophobic residues. Many structure-function studies using different host-derived AMP templates (e.g., SAAP-148 derived from LL37) do not necessarily result in generalized structure-function correlations partly because of the diversity in amino acid composition of such templates [Deslouches B et al., 2020; de Breij A et al., 2018]. A plausible explanation is that these natural AMP templates already include amino acids that may not determine antimicrobial functions, as they are not typically hydrophobic or cationic. Instead, their presence in the primary sequences of these natural peptides can be associated with the multifunctionality of AMPs and influence the folding of the primary sequence in a way that partially hinders optimization specific to antimicrobial function [Levast B et al., 2019; Kumar P et a., 2018]. From an antimicrobial standpoint, these factors need to be controlled. To do so, only those amino acids that are typically cationic or hydrophobic were used, which are more relevant to AMP amphipathic structure as determinants of antimicrobial properties. In addition, it was important to explore the well-studied role of W in overcoming many AMP limitations such as susceptibility to salt and serum as well as the lack of in vivo efficacy [Deslouches B et al., 2020]. Further, Arg (R) was retained over Lys because it is the most basic amino acid and shown to be effective in previous studies. It is in this context that the present study reports a systematic assessment of the impact of W on selective potency against the most common MDR bacteria known as ESKAPE pathogens [Deslouches B et al., 2015; Boucher H W et al., 2009]. Toward this goal, multiple W-based libraries of cationic peptide antibiotics (PAX, designated as a distinction from natural and other engineered AMPs) were engineered (de novo), consisting of a total of 86 peptides in addition to several AMP and antibiotic controls. This study elucidates measurable functional changes associated with minor structural differences to rationally achieve selective antibacterial properties in a way that does not appear to critically affect host toxicity. In vitro structure-function correlations resulted in rationally selected PAX candidates that displayed broad efficacy against MDR strains of ESKAPE pathogens, including antibiotic-resistant isolates (AR isolates) from the Center for Disease Control and Prevention (CDC) [CDC. Antibiotic Resistance Threats in the United States, 2013.]. As proof of concept, one of the selected PAX was tested systemically and demonstrated the ability to mitigate an otherwise lethal infection induced by an MDR strain of Pseudomonas aeruginosa or Klebsiella pneumoniae in mice.

This study began with the design of a series of AMPs based on differential numbers of W residues (e.g., 3, 4, or 5 W residues, Table), charge, and peptide length. Exclusive use of W in the hydrophobic domain may enhance both antimicrobial functions and host toxicity due to its high hydrophobicity and its bulky indole ring [Deslouches B et al., 2020; Deslouches B et al., 2005]. Thus, to balance the nonselective membrane-seeking property of W, Val (V) was designated instead of Phe, Leu, or Ile to complete the hydrophobic domain because of its substantially lower hydrophobicity than that of W. Depending on the number of W residues, the design started with a minimum length of 8 (4R+4W) or 10 (5R+5W or 6R+3W+1V) residues (Table 1) using helical wheel projections to deduce the primary sequences as idealized helices with high hydrophobic moments, which is a measure of amphipathicity (FIG. 1A) [Gautier R et al., 2008]. The length and charge of the peptides was incrementally increased by adding 1R and 1V at a time to each template until a maximum length of 24 (the 3W+4W templates) or 18 (the 5W template) residues was reached (Table 1). The 5-W template was limited to 18 residues in length based on previous observations that increasing the number of W in longer peptides (e.g., 24 residues) markedly increased host toxicity [Deslouches B et al., 2005; Deslouches B et al., 2015; Deslouches B et al., 2013]. This study develops a framework allowing the identification of highly potent and safe peptides that are as short as possible.

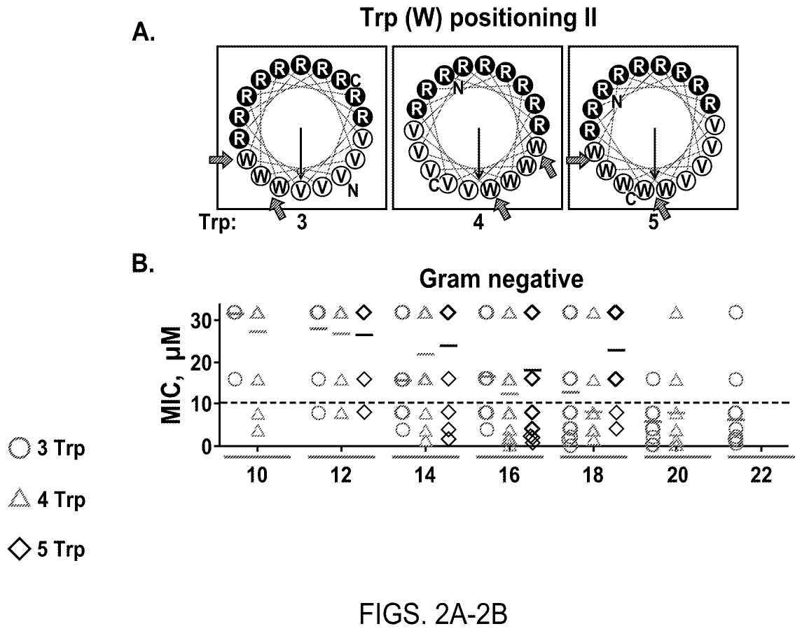

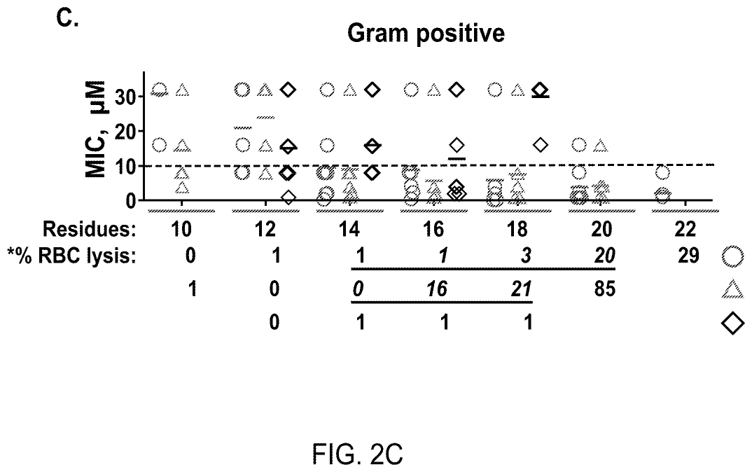

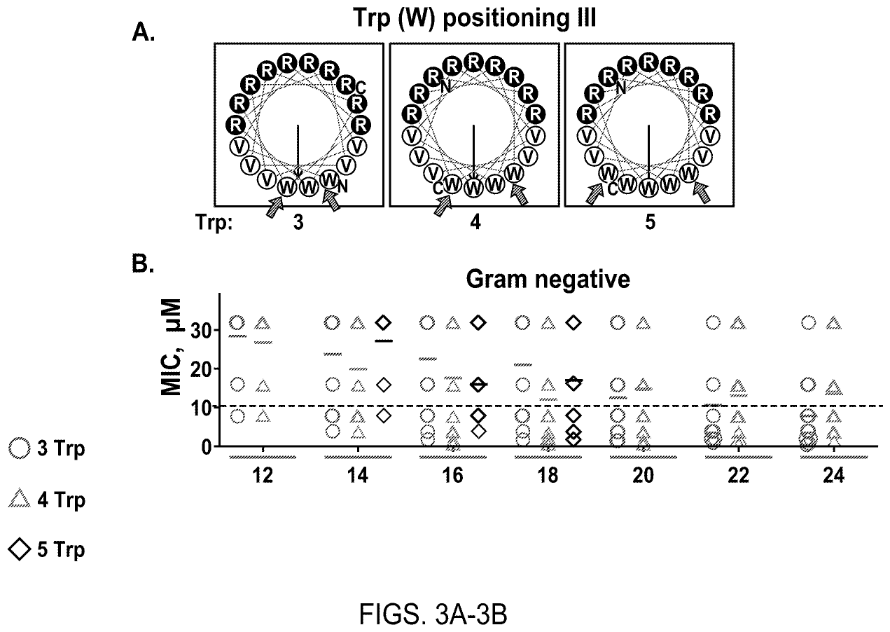

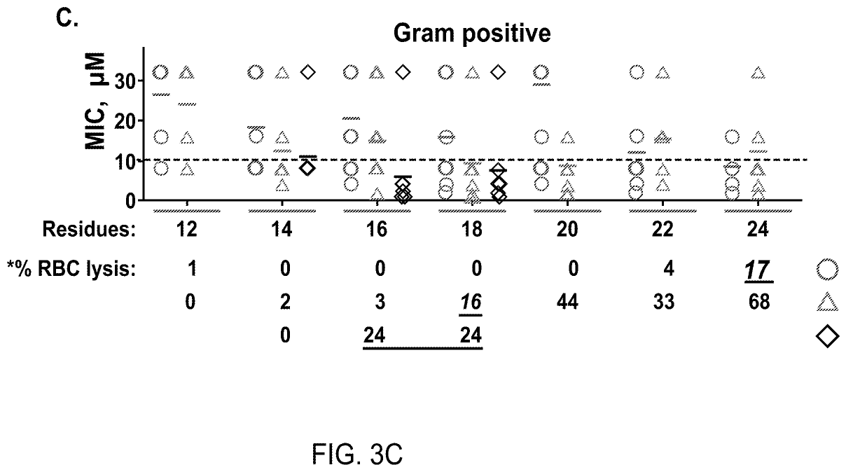

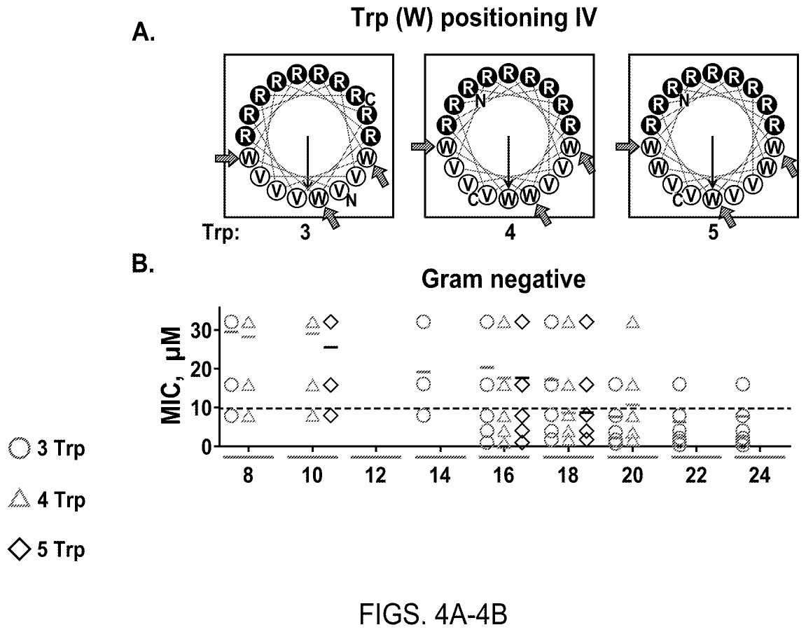

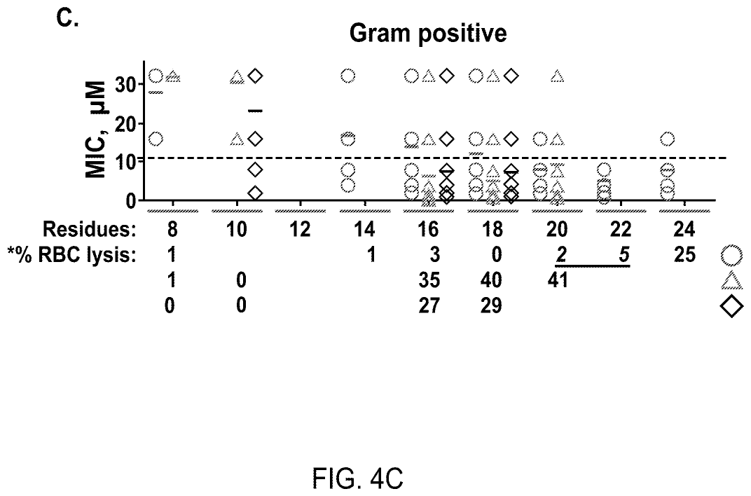

To control for the potential influence of the positions of W residues, W-positional analysis was used to complement the first PAX library (Table 1) with three additional libraries (Tables 2-4), resulting in a total number of 86 de novo-engineered PAX. They were distinguished herein as libraries 1, 2, 3, and 4. In library 1, the W residues are placed on both sides of the hydrophobic-hydrophilic interface of the helical wheel projections of the peptides (FIG. 1A). In library 2, the W residues were asymmetrically aggregated at the hydrophilic-hydrophobic interface (FIG. 2A, Table 2). Library 3 was generated by placing the W residues as far as possible from the hydrophilic domain of the helical wheel projections (FIG. 3A, Table 3). In library 4, the W residues are staggered or equidistantly scattered throughout the hydrophobic domain (FIG. 4, Table 4). Of note, W positions are restricted by the number of W residues and the length of each PAX. Further, there are minor positional variants of PAX of the same length in a particular library (Tables 1-4).

As controls, W-containing AMPs were used such as indolicidin and tritrpticin [Rozek A et al., 2000; Nguyen L T et al., 2011]. The LL37-derived SAAP-148 is also included [de Breij A et al., 2018], one of the most recently engineered AMPs with remarkably broad antibacterial activity and only moderate red blood cell (RBC) lysis at 16 μM. Hence, an internally controlled system was designed using peptides that differ from each other in length by only two residues and by a charge of +1 (Tables 1-4) as well as both engineered and natural AMPs that are rich in W content for additional controls (FIG. 9). A well-known AMP (L-K6L9) with no W content was also included [Braunstein A et al., 2004] in addition to appropriate antibiotic controls including colistin, depending on the bacterial test organism. While the helical wheel projections are represented in FIGS. 1-4 only by 18-residue peptides, every single PAX longer than 8 residues was modeled as a cationic amphipathic helix using the online helical modeling software heliquest [Gautier R et al., 2008].

Next, all peptides were initially screened for antibacterial potency against an MDR panel of gram-negative (GNB) and -positive (GPB) bacterial isolates classified as ESKAPE pathogens, which was previously used in published studies [Di Y P et al., 2020; Deslouches B et al., 2015; Boucher H W et al., 2009]. Within any series of peptides with constant W content, increasing the length resulted in a gradual decrease in mean MIC (library 1) toward optimal activity at a particular length against both GNB (FIG. 1B) and GPB (FIG. 1C). The MIC or minimum inhibitory concentration is the minimum drug concentration that results in no detectable bacterial growth, indicated by a horizontal growth kinetic curve with bacterial growth measured every hour. Mean MICs are lowest at 20-24 AA residues (r) for PAX of 3W, at 16r for PAX with 4W, and 14-18r for 5W peptides. Generally, once the minimum length achieving the lowest MIC (minimum optimal length or MOL) is reached, there was no additional gain in antibacterial activity (FIGS. 1B and 1C) with further increase in length. Thus, we were able to identify a precise range of MOL against the 6 organisms composing the ESKAPE pathogens [Boucher H W et al., 2009]. This structure-MIC trend remained consistent across libraries 1-4 (Tables 1-4 and FIGS. 1-4), although the MOL was both dependent on W content (within a library) and positions (across libraries). Activities were typically 2-fold lower (mean MIC, 2-fold higher) against GNB compared to GPB, although a 2-fold variation in MIC is not unusual in our growth inhibition assays. Thus, the data indicate broad activity of PAX against both GNB and GPB with consideration given to MIC<10 μM for initial peptide selection.

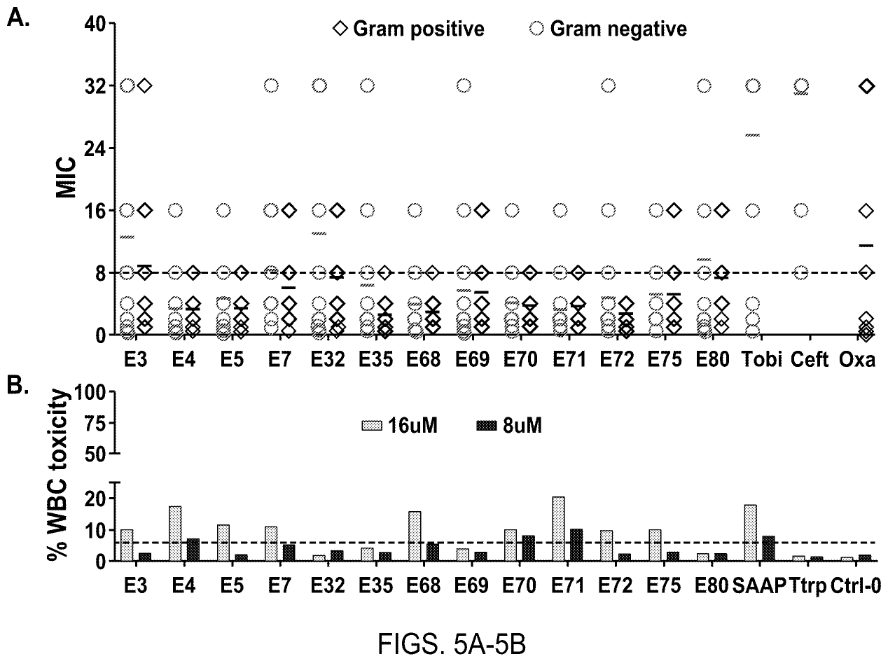

Similar to structure-MIC correlations, all peptides were examined for lytic effects on red blood cells (RBCs) as a primary measure of toxicity to mammalian cells and PAX selection. The percent RBC lysis at maximum test concentration (32 μM, FIGS. 1C, 2C, 3C, and 4C, bottom panels) was shown to increase gradually with peptide length, although the minimum length required for optimal MIC usually corresponded to only minor RBC lysis (<25%, an important criterion for PAX selection). This lag in RBC lysis compared to antibacterial potency at the MOL indicates the effective uncoupling of antibacterial activity and host toxicity or high antibacterial selectivity based on MIC<10 μM and RBC lysis <25% at 32 μM. Thus, the MIC-based MOL was adjusted by considering both MIC and RBC lysis for PAX selection for advancement. That is why only thirteen broadly active PAX met the selection criteria for further investigations (FIG. 5). Library 1 resulted in the selection of E4 (22r in length), E7 (20r), and E72 (22r) as 3W PAX and E32 (14r), E35 (16r) as 4W PAX; library 2: E68-70 (3W, 20r), E71 (3W, 22r), E75 (3W, 22r), E80 (4W, 16r); library 3, only E3 (3W, 24r); library 4, only E5 (3W, 22r). There was no final selection of 5W PAX, as increasing the number of W beyond 4 did not result in appreciable gain in activity and safety. Thus, libraries 1 (FIGS. 1) and 2 (FIG. 2) produced the highest number of selected PAX (a total of 11 of the 13 selected peptides), based on mean MIC<10 μM and <25% RBC lysis at maximum test concentration. The strain-specific MICs of the selected PAX against the MDR strains used in FIGS. 1-4 are shown in Table 5. Notably, colistin was inactive against the GPB strains as expected, although KPA5 is the only GNB strain to display a colistin-resistant phenotype. Colistin cross-resistance of KPA5 was not observed among the selected PAX, with MIC varying from 1 to 8 μM (Table 5).

FIG. 9 showed a lack of activity of the natural AMPs indolicidin and tritrpticin (mean MIC>10 μM) against the recalcitrant panel of bacteria, which is partly due to the contextual activity of these AMPs in a stringent test medium (cation-adjusted Mueller-Hinton broth, MHB2). The engineered AMP K6L9 (with no W content) was not as effective against the test bacterial panel (mean MIC>10 μM) as the selected PAX and demonstrated a high lytic effect (>50% at 32 μM) on human RBCs. In sharp contrast, the 24-residues SAAP-148 (with 2-W content) was broadly active (mean MIC<10 μM) against both GNB and GPB, although the engineered peptide demonstrated >50% RBC lysis at the maximum test concentration of 32 μM. Notably, colistin demonstrated the lowest mean MIC, although the breakpoint for resistance is limited to approximately 2 mg/L or ˜2 μM due to its nephrotoxicity [Bardet L et al., 2018; Nation R L et al., 2016; Horcajada J P et al., 2016]. Taken together, the data indicate that the structure-function framework constitutes a rational design system for distinguishing highly active and short peptides with minimal RBC lysis from those that are highly erythrolytic (>25% lysis at high concentrations) above the MOL.

Based on these primary structure-function correlations, we further examined the 13 selected PAX for broad antibacterial activity against MDR clinical strains of ESKAPE pathogens and E. coli provided by the CDC (AR isolates panel), with the strains and their resistance profiles presented in Table 6. Similar to the primary screening, the advanced testing utilized growth inhibition assays in MHB2. The majority of the selected PAX remained broadly active against both GNB and GPB AR isolates from the CDC (mean MIC<10 μM for most selected PAX, FIG. 5A) with slightly lower mean MICs against GPB, consistent with the initial observation with the ESKAPE panel shown in FIGS. 1-4. Similar to the Table 5, The strain-specific MIC data are tabulated in Table 7. As a progression from the primary screening, toxicity to freshly isolated human white blood cells (WBC) was also examined to further select for lower toxicity among these broadly active peptides. The selected PAX at a maximum test concentration of 16 μM displayed a toxicity level against WBC (measured by flow cytometry) that was consistently low or negligible (typically <10%) (FIG. 5B). For the RBC lysis and WBC toxicity assays, it was not practical to show all the data points for lower test concentrations because of the high number of test peptides, and toxicity was negligible in most cases at these test concentrations. Unlike the first MDR panel (Table 5), some level of cross-resistance was observed among 8 PAX for KP542 and 5 PAX for PA229 (MIC>16 uM) (Table 7). To further investigate cross-resistance to colistin, several additional colistin-resistant strains were identified according to CDC data for comparison of several of the selected PAX. Interestingly, the PAX displayed MICs in the range of 1 to 8 μM (Table 8). These data indicate that, in contrast to the two cases of cross-resistance with colistin (Table 7), resistance to colistin can be overcome by structurally optimized PAX. In that context, a PAX cocktail can be an effective option to obtain broad coverage against extensively resistant GNB isolates that are also recalcitrant to colistin treatment. Importantly, PAX activity extends across both GNB and GPB, in contrast to colistin activity only against GNB. Next, a single PAX and an extensively resistant bacterial strain were chosen (PA239, Table 6) to conclude the current investigation with mechanism and proof-of-concept experiments. Similar to the other selected PAX, E35 displayed broad activity against both test panels of GNB and GPB (FIGS. 1 and 5) and remained active against several colistin-resistant strains. E35 was ultimately chosen because it displayed the shortest MOL (at 16r), as length was an important structural parameter in our structure-function studies. However, any of the remaining selected PAX may display a lower MIC than that of E35 against specific strains or vice versa.

### Example 2. PAX E35 Kills P. aeruginosa by Perturbing the Bacterial Membrane

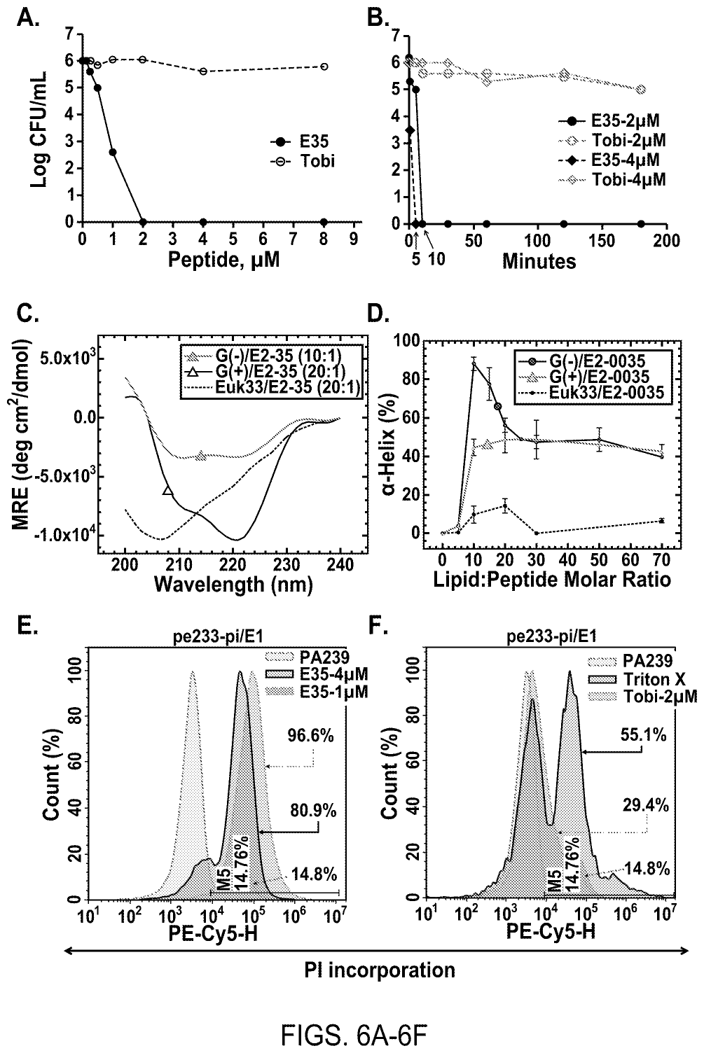

While multiple antibacterial mechanisms have been demonstrated for a variety of cationic AMPs [Di Y P et al., 2020; Kumar P et al., 2018; Bechinger B et al., 2017; Deslouches B et al., 2016; Gee M L et al., 2013; Brogden K A et al., 2005], membrane perturbation is the most predominant mechanism of helical AMPs [Roncevic T et al., 2019; Lohner K et al., 2017]. To test whether PAX follow this common mechanism, the experiment initially examined the bactericidal activity of the top selection, PAX E35, against the MDR P. aeruginosa strain PA239 (resistant to all 12 test antibiotics, including colistin, Table 6). Bactericidal properties were assessed using a medium called FMP (FBS, 15%; MHB2, 25%; PBS, 60%) that is more physiologically relevant than MHB2 with the inclusion of saline and fetal bovine serum, a known inhibitory condition for many AMPs [Di Y P et al., 2020; Deslouches B et al., 2005]. The peptide killed all bacterial cells at 2 μM concentration, whereas tobramycin did not display any substantial bacterial killing within the 3 h of incubation as expected (FIG. 6A). In addition, E35 killed 2 logs of the bacterial cells within the first minute of treatment at 2 μM and 4 μM, compared to the non-effective tobramycin (FIG. 6B). No bacterial cells survived E35 treatment after 5-10 minutes of incubation. However, taken together with real-time video imaging, what appeared to be complete bacterial killing within 5-10 minutes (FIG. 6B) was partly irreversible membrane disruption that eventually resulted in cell death. Consistent with these bactericidal assays, during early exposure to the peptide PA239 at high inoculum (5×10⁸ CFU/mL) and 8 μM concentration, the bacterial cells displayed substantial incorporation of propidium iodide (PI), which is impermeable to intact cell membrane. In fact, during the first 15 minutes of exposure to E35, bacterial cells appeared to be disintegrated as DNA-containing subcellular particle aggregates. Also noteworthy was the presence of many PI-incorporated cells that retained swimming motility. These bacterial cells continued to swim even after more than an hour, indicating that a substantial number of the E35-exposed bacterial cells were enumerated as dead in the killing kinetic experiment within 5-10 minutes only because their cell membranes were irreversibly damaged by the peptide and, therefore, cannot survive on an agar plate overnight. Certainly, many E35-treated cells died within minutes as shown by scattered aggregates of dead cells by 15 minutes. Thus, despite massive PI incorporation, real-time video imaging in bright field showed a substantial proportion of the PI-incorporated PA239 cells were still alive (swimming motility) after 1 hour of peptide treatment. In sharp contrast to E35-treated cells, mock-treated and tobramycin-treated PA239 cells displaying swimming motility were not visible in the PI channel (blank screen) because they did not demonstrate any markedly detectable intracellular PI. These results indicate that the membranes of these bacterial cells remained uncompromised despite background cell death observed from the results of bactericidal assays (e.g., FIGS. 6A and 6B).

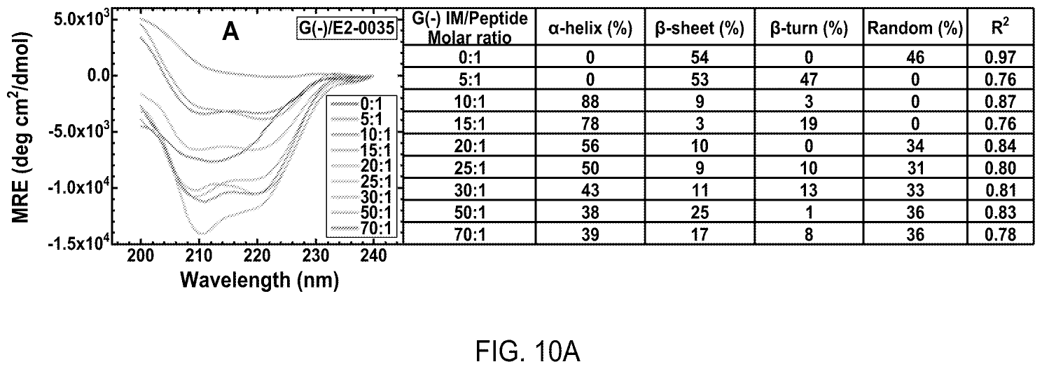

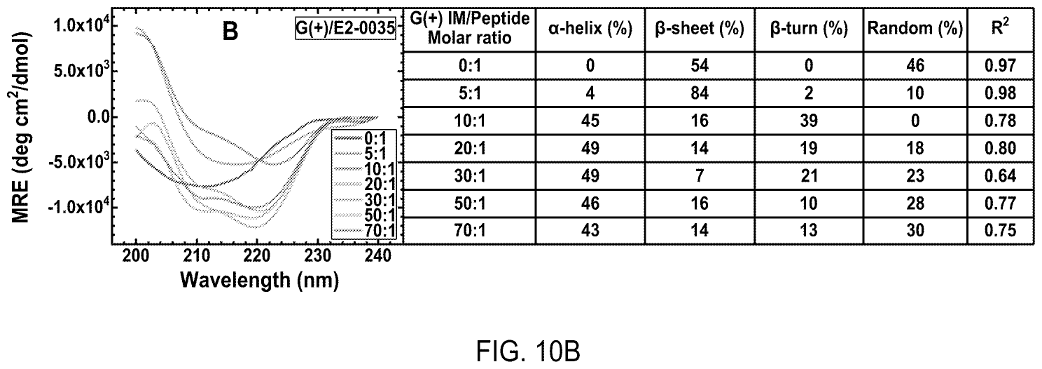

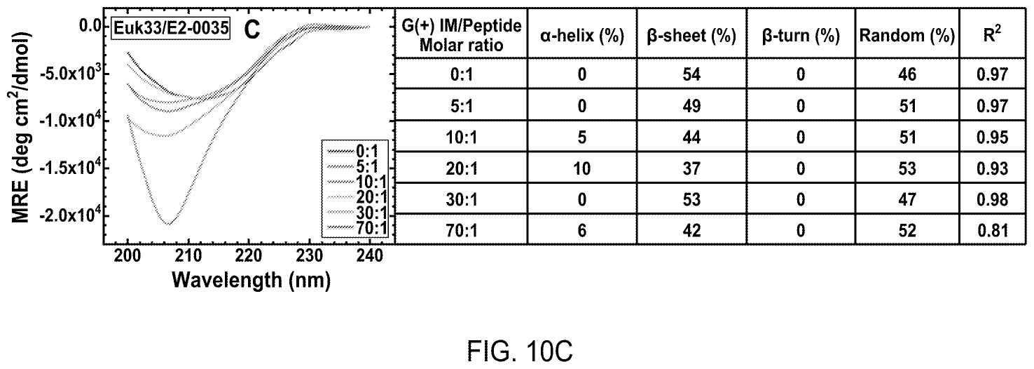

Next, membrane mimics of GPB, GNB, and eukaryotic cells were interrogated for interactions with E35 to examine their impact on the folding of the selected PAX E35 using circular dichroism [Deslouches B et al., 2005; Heinrich F et al., 2020]. When comparing the lipid-to-peptide molar ratio at which the highest helical content was observed (FIG. 10), the CD spectra indicate that the E35 peptide has a higher propensity to fold into a helical structure in the presence of bacterial compared to eukaryotic membrane mimics (FIGS. 6C and 6D). These results are consistent with the broad antibacterial activity of PAX compared to toxicity, or lack thereof, to mammalian cells (FIGS. 1 and 5). Surprisingly, the helicity was substantially higher for GNB compared to GPB membrane mimics, although the MICs for GPB were consistently 2-fold lower than those for GNB. Thus, the differential helicity appears partly inconsistent with the differential anti-GNB and anti-GPB activities. Of note, the membrane mimics may not accurately reflect the differences in lipids displayed by the surfaces of GNB and GPB. It was also confirmed that this selective interaction with bacterial membranes resulted in killing principally by membrane perturbation, using flow cytometry to detect cell death by PI incorporation. As shown in FIG. 6E, the bacterial cells incorporate 81% and 97% PI in a concentration-dependent manner, 1 μM and 4 μM respectively. In sharp contrast, only 29% PI incorporation was observed when cells were treated for 3 hours with tobramycin (2 μM) compared to 14% incorporation of mock-treated cells. Bacterial cells treated with 1% triton X-100 displayed 55% PI incorporation (FIGS. 6E and 6F). Importantly, these cells were treated for 3 hours, which may explain more background PI incorporation compared to PI detected by real-time video imaging in cells treated with drugs for a shorter period.

### Example 3. Therapeutic Efficacy in P. aeruginosa and Klebsiella Models of Infection in Mice

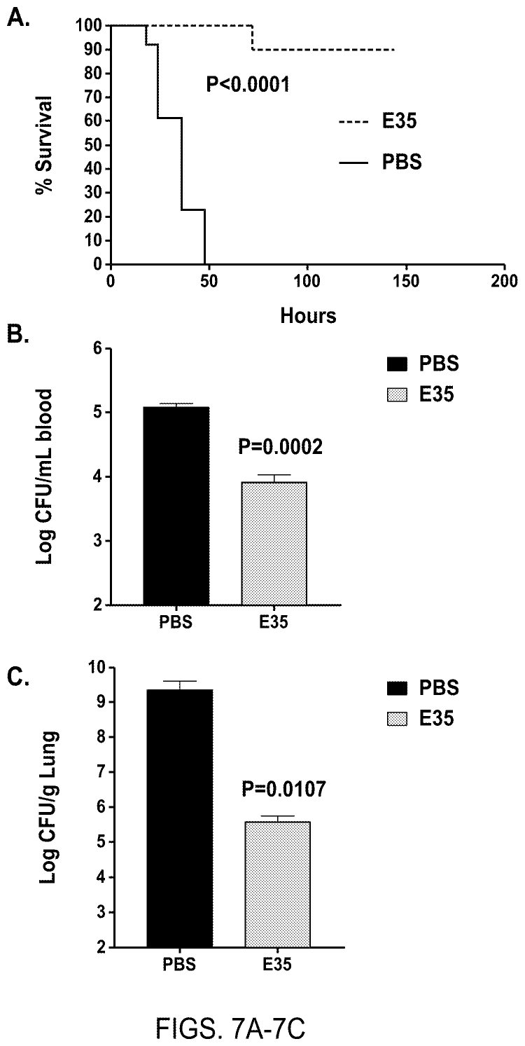

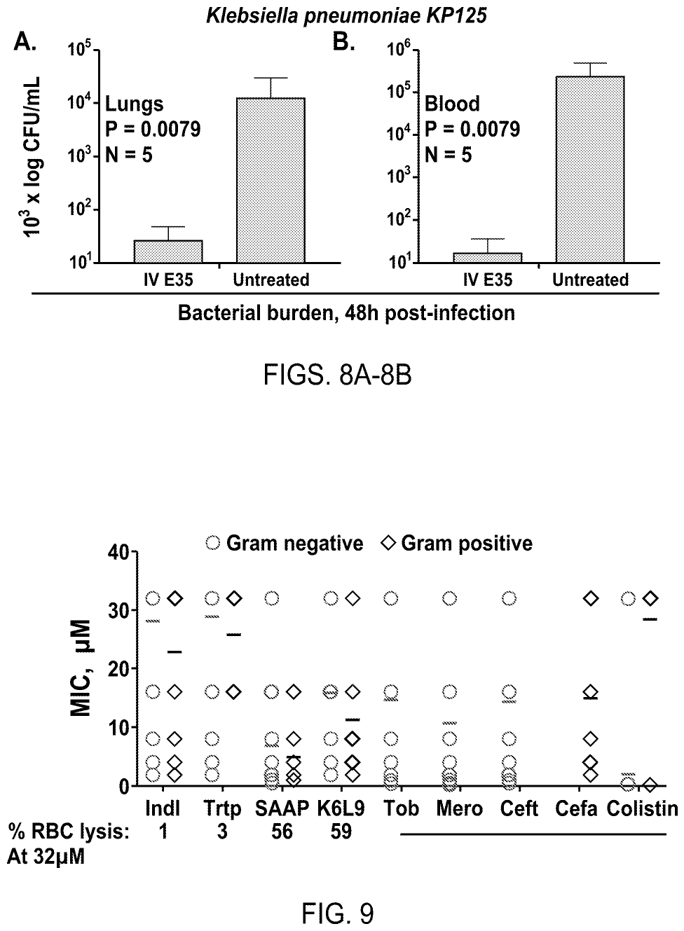

An important question is whether the in vitro activity is reflective of the therapeutic efficacy of selected peptides in animal models. To address this question, whether PAX E35 can be tolerated in mice at a high systemic dose (maximum tolerated dose, MTD) was determined. 15 CD-1 mice were injected via the tail vein with various doses up to 45 mg/kg (mpk). The mice were able to tolerate the peptide at up to 30 mpk. To confirm the MTD, 7 mice were given saline or E35 systemically at 30 mpk, and no apparent morbidity and mortality were. Next, PAX E35 for efficacy against PA239 was examined in mice. The minimum dose of PA239 lethal to CD-1 mice if injected in the intraperitoneal (IP) cavity was determined (minimum bacterial lethal dose, MBLD). The MBLD for this MDR strain was found to be 2×10⁷ CFU in <20 g CD-1 mice. The mice typically succumb (100% mortality) to P. aeruginosa sepsis within 18-48 h (FIG. 7A) in the absence of effective treatment. In sharp contrast, almost all (12/13) mice treated systemically with E35 (5 mpk) approximately one to two hours after bacterial exposure displayed no signs of infection when monitored up to 7 days (FIG. 7A). The effects of E35 on bacterial burden were examined at 5 h post-treatment. As shown in FIG. 7B, bacterial burden in the blood was reduced by 10-fold compared to mock-treated mice. A 10,000-fold reduction in lung bacterial load was also observed (compared to mock-treated mice). Thus, using a strain that is resistant to 12 standard-of-care test antibiotics (Table 5), including colistin, demonstrated that the E35 peptide has the property to rapidly reduce bacterial burden within hours after the administration of a single systemic dose without affecting the ability of the animals to eliminate the remaining bacterial cells. Similar results were demonstrated when immune-compromised mice infected IP with K. pneumoniae (KP125, MIC in Table 8), with bacterial burden reduced by>3 logs in both the lungs (FIG. 8A) and the blood (FIG. 8B) after daily doses of 4 mpk of E35 for two days (FIG. 8). Taken together, these results indicate that this framework can be used for elucidating the determinants of activity through structure-function correlations for the identification of PAX that are safe in vitro and in vivo and able to overcome resistance to traditional antibiotics.

This report shows the development of a rational framework for the design of cationic amphipathic peptides with the property to overcome antibiotic-resistant bacterial infections. We showed that, given a de novo template containing W and R as the principal amino acids (AA) and varying incrementally in length by a single R (a charge of +1) and V residue at a time, it is possible to define a W content-dependent range of MOL for activity at low micromolar concentrations against MDR bacteria. The data revealed that the MOL was the lowest (16r, E35) when the W content was kept at 4 residues, and most selected peptides demonstrated moderate to negligible toxicity to freshly isolated human RBC and WBC at high test concentrations. At comparable length, the 4-W PAX series tends to display higher toxicity to mammalian cells than that of 3-W PAX. However, this toxic effect is compensated by a much shorter MOL of the 4-W at which only minor toxicity to RBC and WBC was observed at the highest test concentration, which is the key rationale for choosing E35 among the selected PAX to conclude this study. It is at the MOL that antibacterial property and toxicity to mammalian cells are typically uncoupled, thereby effectively enhancing the antibacterial selectivity or increasing the therapeutic index. Importantly, activity can be reduced when the peptides are elongated beyond that MOL, which is consistent with previous observations [Deslouches B et al., 2005; Deslouches B et al., 2015]. Additionally, the positions of the W residues can affect both antibacterial function and toxicity to host cells [Rekdal O et al., 2012], which explains the differential yield in selected PAX favoring libraries 1 and 2 over libraries 3 and 4. Library 2 produced the highest number (6 of 13) of selected PAX with all W residues positioned at a single side of the hydrophobic-hydrophilic interface. Library 1 is based on the symmetric (or near-symmetric) distribution of the W residues on both sides of hydrophobic-hydrophilic interface and resulted in the selection of 5 PAX, including E35. Thus, the W-positional analysis revealed that positioning the W residues at the hydrophilic-hydrophobic interface is optimal, regardless of symmetry, for the selection of PAX that are highly potent with minimum risks of host toxicity at the MOL. The examination of toxicity avoided the use of immortalized cell lines to determine toxicity to host cells due to the fact that AMPs can be selectively toxic to tumor cells compared to primary cells [Deslouches B et al., 2017]. In that context and beyond the scope of this work, this framework can also be used to selectively distinguish PAX that are highly toxic to tumor cells at concentrations that are nontoxic to primary cells, which can be a starting point for screening cationic anticancer peptides. The data herein also revealed evidence for a bactericidal mechanism due to membrane disruption. Specifically, flow cytometry and real-time video imaging studies demonstrate that E35 killed bacteria via direct disruption of the membrane of the bacterial cells. Importantly, real-time video imaging was critical to the correct interpretation of the killing kinetic data, indicating that the early (5-10 minutes of peptide treatment) cell death was partly due to irreversibly compromised membranes of the cells, which did not growth on agar plates overnight for inclusion in the bacterial enumeration. In addition, real-time video imaging also revealed cell death during early exposure to the peptide demonstrated by the aggregates of PI-bound subcellular particles (in this case DNA). Thus, live-cell fluorescence microscopy complemented the flow data by revealing massive cellular damage (subcellular particles) that could not be detected by flow cytometry in addition to PI incorporation by live cells with compromised membranes. Of note, the high bacterial inoculum used in the mechanistic studies, compared to that used in the antibacterial assays, required a proportionally elevated PAX concentration, which was consistent with the sensitivity of the flow and video imaging.

Recent advances in the field of AMPs showed that modifications of host-derived AMP templates can result in highly potent peptides with therapeutic efficacy in mice. In particular, the LL37-derived peptides (containing 2 W residues) SAAP-148 and ZY4 demonstrate broad antibacterial activities [Mwangi J et al., 2019; de Breij A et al., 2018]. SAAP-148 was used as one of the peptide controls because of its broad and potent activity against both GNB and GPB, which was confirmed by our preliminary screening data. However, while published data show a peptide with moderate lytic effects on the human RBCs at 16 μM, the data here indicate that SAAP-148 was more erythrolytic than published data indicate. One explanation is that the maximum test concentration was higher in the correct study, and sensitivity of both studies are not comparable. Moreover, the topical application as an ointment in mice to combat biofilm-related skin infection may not be highly relevant to RBC lysis, and the toxicity to WBC was lower than its erythrolytic effects. Another recent advance in AMP design is demonstrated by another LL37-derived peptide ZY4 [Mwangi J et al., 2019]. This peptide is also broadly active and highly potent against bacteria. Unlike the skin ointment application of SAAP-148, ZY4 is given IP in multiple doses using a murine IP model of bacterial infection. While the peptide was not examined for its impact on mouse survival, its effects on reducing bacterial burden were substantial and significant. Although the IP administration is certainly an important advance from the skin application of SAAP-148, similar to other examples of engineered AMPs [Mourtada R et al., 2019], both bacteria and the drug were given IP and, therefore, followed the same path. Thus, it is not clear whether the efficacy of the peptide necessitated efficient absorption and distribution to tissues to suppress the infection or if the peptide acted primarily at the site of delivery. In contrast, the instant study demonstrated that one of the selected PAX E35 works systemically against the IP-injected MDR P. aeruginosa by rapidly reducing the bacterial burden in blood and in lung tissue. Systemic efficacy is also demonstrated in immune-compromised mice infected IP with K. pneumoniae, also resistant to colistin. This demonstration of efficacy in two different mouse models is highly relevant to standard of care in hospitalized patients, who typically require IV injection to suppress MDR-related bacteremia or sepsis. Importantly, the peptide was effective either at a single dose of 5 mpk (immune-competent model) or 2 doses (once daily) of 4 mpk (immune-compromised model), with an MTD of 30 mpk. The efficacy against the two colistin-resistant isolates was an important consideration, although cross-resistance with E35 was observed with two (PA229 and KP542) of the 11 colistin-resistant strains. Notably, some selected PAX (e.g., E4, E68, E70, E71) displayed no cross-resistance at all, which underscores the need for an extensive comparative study of the selected PAX against MDR isolates that are highly resistant to colistin.

While W, length, and charge were previously used as determinants of toxicity and antibacterial properties [Deslouches B et al., 2005; Deslouches B et al., 2013; Rekdal O et al., 2012], this study is remarkably impactful for the following reasons. (A) It is the most systematic and extensive structure-function study of its kind utilizing 4 libraries consisting of 86 de novo designed peptides examined simultaneously for MIC and toxicity as a function of charge, length, as well as W content and positioning. (B) It provides a rational framework for structure-function correlations using W- and R-rich AMPs of helical tendencies. (C) Scientific rigor is indicated by only slight differences between PAX of similar W content, with the inclusion of engineered and natural AMPs in addition to standard-of-care antibiotics as controls. (D) This design system can be used as an iterative framework for testing any combination of cationic and hydrophobic amino acids for structure-function correlations, reducing the need for trial and error. (E) The bacterial panels are highly reflective of common clinical MDR strains, using clinical isolates from local medical center and the CDC. (F) The effects was demonstrated in two infection models with distinct routes of infection (IP) and treatment (systemic), despite that P. aeruginosa was not the most susceptible target (based on mean MIC) of the peptide among the ESKAPE pathogens, which indicates many applications of the selected peptides. (G) Efficacy was demonstrated in both immune-competent and-compromised mice.

This study provides: (1) a system for fundamental discoveries of cationic peptides with enhanced safety and efficacy, which can be extended to peptide mimics and other structural classes; (2) a clear departure from topical to systemic use of engineered AMPs.

### Example 4. Materials and Methods

Reagents. Bacterial media cation-adjusted Mueller Hinton Broth (MHB2) was obtained from Millipore Sigma (St Louis, MO, USA). Fixable live/dead stain and Propidium iodide (PI) were purchased from Thermo-Fisher (Waltham, MA USA). All peptides were obtained in lyophilized form of 10 mg in a 1.5 mL vial from Genscript (NJ, USA) with HPLC/MS spectra corresponding to each designed primary sequence. Each peptide vial was labeled with a code name (E followed by a number) without sequence identity, including peptide controls (e.g., indolicidin) [Jiang S et al., 2019], and was dissolved by adding 1 mL filter-sterilized PBS to each peptide (10 mg/mL). The comprehensive names, which were used during the design of libraries, were only utilized after the analysis of the data.

Bacteria. Bacterial clinical isolates, used for initial screening and in previous studies [Greber K E et al., 2017], were anonymously provided by the clinical microbiology laboratory of the University of Pittsburgh medical Center. The panel used for secondary screening was from CDC (Table 4), with resistance profiles data. Bacteria are stored in lock-secured −80° C. freezer and typically retrieved by obtaining single colonies on agar plates prior for subsequent liquid broth cultures. Suspensions of test bacteria were prepared from log phase of growth by diluting overnight cultures at 1:100 with fresh cation-adjusted MHB (MHB2, Millipore Sigma, USA) and incubating for additional 3-4 hours. Bacteria were spun at 3000×g for 10 minutes and the pellet resuspended in test condition media (Millipore Sigma, USA) to determine bacterial turbidity using a Den-1B densitometer (Grant Instruments, USA) at 0.5 McFarland (unit of bacterial density) corresponding to 108 CFU/mL.

Antibacterial assays. To examine antibacterial activity, this study used minor modifications of a standard growth inhibition assay endorsed by the Clinical and Laboratory Standards Institute (CLSI), as previously described [Greber K E et al., 2017]. Bacteria were incubated with each of the indicated peptides in MHB2. The bacterial cells were kept in an incubator for 18 hours at 37° C., which is linked to a robotic system that feeds a plate reader every hour with one of 8×96-well plates. This set-up allows the collection of growth kinetic data at A 570 (absorbance at 570 nm) to examine growth inhibition in real time (BioTek Instruments, USA). MIC (minimum inhibitory concentration) is defined as the peptide concentration that completely prevented bacterial growth, which shows a flat (horizontal line) bacterial growth kinetic curve at A570. To assess bactericidal properties of the peptide, the assay was modified by using 15% fetal bovine serum (FBS, for a more physiologically relevant test condition) mixed with 25% MHB2 and 60% PBS. Bacterial cells (colony forming unit/mL or CFU/mL) were enumerated after incubating treated bacteria for 3 hours and plating serially diluted samples on broth agar medium at 37° C. overnight. MBC (minimum bactericidal concentration) was determined as the peptide concentration at which complete bacterial killing was observed [Rozek A et al., 2000]. For killing kinetics, we slightly modified the bactericidal assay to examine bacterial killing at the MBC (or 2×MBC) as a function of time for up to 3 hours. Data were analyzed using GraphPad Prism software.

Determination of toxicity to mammalian cells. Toxicity to primary cells was examined using human red blood cells and peripheral mononuclear cells (PBMC or white blood cells) as previously described [Lipsky B A et al., 2008]. Briefly, RBCs and WBCs were separated by histopaque differential centrifugation using blood anonymously obtained from the Central blood Bank (Pittsburgh, USA). For RBC lysis assay, the isolated RBCs were resuspended in PBS at a concentration of 5%. The peptides were serially diluted 2-fold in 100 μL of PBS prior to addition of 100 μL of 5% RBC to a final dilution of 2.5% RBC in order to ensure that the absorbance (570 nm) of hemoglobin does not surpass the maximum detectable absorbance of the plate reader. In parallel, the RBCs were treated with water at different concentrations to generate a standard curve of RBC lysis that was be used to determine the percent RBC lysis in the test samples. Experiments were independently conducted by three technicians to ensure reproducibility.

Human WBCs were treated with each selected peptide in RPMI and 10% FBS and incubated for 1 h at 37° C. The cells were then immediately washed with PBS at 1000 g for 7 minutes, while in a round-bottom 96-well plate. After resuspension in PBS, fixable blue live/dead stain from Life Technologies was added according to the manufacturer's instructions. The cells were again washed and resuspended in PBS to remove nonspecific stain and then fixed with 4% formaldehyde (Thermo-Fisher Scientific) for 1 hour. After washing with PBS, the samples were stored at 4° C. overnight (in the dark) before examination by flow cytometry using the Novocyte flow cytometer (Agilent, USA). Peptide-treated samples were compared with untreated control for incorporation of the dye, and data were analyzed using the Novocyte analytical software. Dye incorporation was quantified as Percent toxicity directly determined by distinguishing live from dead populations [Martin E et al., 1995; Deslouches B et al., 2015], which was plotted using GraphPad (Prizm software).

Interaction with membrane mimics by circular dichroism (CD) spectroscopy. Synthetic lipids were purchased from Avanti Polar Lipids (Alabaster, AL) in the lyophilized form and used as received. Cholesterol was from Nu-Chek-Prep (Waterville, MN). HPLC grade organic solvents were from Sigma-Aldrich (St. Louis, MO). Lipid membrane mimics were prepared by mixing stock solutions in chloroform to achieve the following lipid:lipid molar ratios. Gram-negative inner membrane: (7:2:1) 1-palmitoy-2-oleoyl-sn-glycero-3-phosphoethanolamine (POPE): 1-palmitoyl-2-oleoyl-sn-glycero-3-phospho-(10-rac-glycerol) sodium salt (POPG): 10,30-bis-[1,2-dioleoyl-sn-glycero-3-phospho]-sn-glycerol sodium salt (TOCL, i.e., cardiolipin); Gram-positive cellular membrane: (6:1.5:1.5:1) POPG: 1,2-dioleoyl-3-trimethyl-ammoniumpropane chloride salt (DOTAP): POPE: TOCL; Euk33: (5:1:3) 1-palmitoyl-2-oleoyl-sn-glycero-3-phosphocholine (POPC):POPE: cholesterol. Lipid stock solutions were vacuum dried, then hydrated in 15 mM PBS by thermal cycling and vortexing to produce 20 mg/ml multilamellar vesicles, which were then extruded through 500 Å nucleopore filters 21 times to produce 15 mg/ml unilamellar vesicles (ULVs) (determined gravimetrically). ULVs were added to 3 ml 10 μM peptide in 15 mM PBS with pH ˜7 to produce the following lipid:peptide molar ratios: 5:1, 10:1, 20:1, 30:1, 50:1, 70:1. Data were collected in quartz cuvettes with a 1 cm pathlength using a Jasco 1500 CD spectrometer. Samples were allowed to equilibrate for 5 minutes at 37° C., and then scanned from 200 to 240 nm 20 times and the results were averaged. The parameters for scanning were at a speed of 100 nm/min, a step size of 1.0 nm, a response time of 1 second, a bandwidth of 1 nm, and a sensitivity of 20 mdeg.

Background scans of the same concentration of ULVs were first subtracted from the raw ellipticity traces; sometimes traces were smoothed using adjacent averaging of 2-5 nm. OriginPro was used to carry out a linear least squares fit of the ellipticity traces to four secondary structural motifs representing α-helix, β-sheet, β-turn and random coil [Boucher H W et al., 2009].) This analysis gives a percentage match of each of the secondary structural motifs to the total sample ellipticity. Goodness of fit was evaluated visually and quantitated using an adjusted R2. Various constraints and weighting procedures were used to optimize the fits.

Determination of membrane permeabilization. To examine whether the selected peptides kill their bacterial target mainly by membrane permeabilization, this study used PI incorporation by the MDR strain PA239 (5×10⁸ CFU/mL) quantitated by flow cytometry (Novocyte, USA) or visualized by video imaging using the digital microscope EVOS M7000 (Thermo-Fisher, USA) in bright field or red fluorescence channel. In the flow cytometry experiments [Deslouches B et al., 2015], bacterial cells were either untreated or treated with different peptide E35 concentrations in MHB2 for 1 hour at 37° C. The bacteria-peptide mixture was washed with PBS using an Eppendorf centrifuge at top speed for 7 minutes and incubated with PI in the dark for 15 min at room temperature. Tobramycin was used as a negative control with a 3-hour long incubation. For video imaging, the broth medium was reduced to 20% in PBS (treated or untreated) to decrease the rate of cell division and PI incorporation assessed over time. Video images were recorded in real time using the 40× objective either in bright field or red fluorescence channel.

Mouse toxicity. For all animal experiments, the protocol #18071776 approved by the Institutional Animal Care and Use Committee (IACUC) of the University of Pittsburgh was used, which is consistent with the NIH guide for the care and use of laboratory animals. In a preliminary (pilot) experiment, female CD-1 mice (3 per group) weighing about 20 g were injected different doses of peptides up to 45 mpk via a lateral tail vein (IV) to determine an MTD (dose that results in no apparent morbidity or mortality). To confirm the MTD determined in the pilot study, CD-1 mice (7 per group) were randomized to receive a dose based on the preliminary MTD (IV) or PBS (100 μL). The animals were monitored for up to 7 days for morbidity and survival.

Murine infection treatment models. Three to four week-old female CD-1 mice (<20 g) were purchased from Charles Rivers. P. aeruginosa infection was established by IP injection of 1 to 5×10⁷ bacterial CFU in a small pilot experiment to determine a minimum bacterial lethal dose (MBLD, bacterial dose resulting in 100% mortality). The MBLD was then used to induce infection in the treatment model. Mice were typically treated with PBS or peptide 1-2 hours after bacterial exposure. A peptide dose of 4-5 mg/kg was used in 100 μL PBS. We also used an immunocompromised mouse model using cyclophosphamide injection, 150 mg/kg on first injection and 100 mg·kg daily for 4 consecutive days. K. pneumoniae was injected i.p. on day 6, and treatment was initiated 2-3 hours post-infection and continued daily for 2 days (a total of 2 doses of 4 mg/kg). During the experiments, we monitored the mice for signs of morbidity and survival or mortality (moribund state). Mice were euthanized by CO₂ inhalation either at 5 h (immune-competent mice) or 48 h (immune-compromised mice) post-treatment to determine bacterial burden or when moribund (survival endpoints). Bacterial load in the blood and in total lung homogenates was determined by serial dilution and plating on agar overnight before enumeration as CFU/mL (blood) or CFU/g tissue (lung) [Martin E et al., 1995].

Statistical analysis. The results are represented in graph format as mean±standard deviation displayed by the error bars. For in vitro studies and bacterial burden in tissues, we analyzed differences between test samples for statistical significance using unpaired t test with two-tailed P values or the Kolmogorov-Smirnov test. For survival studies, we used Kaplan Meir analysis with the Log-rank (Mantel-Cox) and Gehan-Breslow-Wilcoxon tests. The error bars in the CD analysis were obtained from several fitting attempts of a single sample, as well as from duplicate samples. P values under 0.05 are considered for statistical significance. The GraphPad software was used for all analyses unless otherwise indicated.

TABLE 1
Primary sequences and physicochemical properties of Library 1 peptides

| | Name in | | Trp | SEQ | | Length | | | |
|---|---|---|---|---|---|---|---|---|---|
| Name | Provisional | Code | (W) | ID NO | Sequence | (r) | Charge | H | μH |
| hPAXI-1 | AMP3I-10 | E23 | 3 | SEQ ID | WWRRVRRRWR | 10 | 6 | 0.191 | 0.859 |
| | | | | NO: 1 | | | | | |
| hPAXI-2 | AMP3I-12 | E19 | 3 | SEQ ID | WWRRVRRRWRRV | 12 | 7 | 0.177 | 0.859 |
| | | | | NO: 2 | | | | | |
| hPAXI-3 | AMP3I-14 | E16 | 3 | SEQ ID | WWRRVRRVWRRVRR | 14 | 8 | 0.166 | 0.851 |
| | | | | NO: 3 | | | | | |
| hPAXI-4 | AMP3Ia-14 | E63 | 3 | SEQ ID | VWRRVRRWWRRVRR | 14 | 8 | 0.166 | 0.832 |
| | | | | NO: 84 | | | | | |
| hPAXI-5 | AMP3Ia-16 | E64 | 3 | SEQ ID | VWRRVRRWWRRVRRRV | 16 | 9 | 0.159 | 0.79 |
| | | | | NO: 85 | | | | | |
| hPAXI-6 | AMP3I-16 | E13 | 3 | SEQ ID | WWRRVRRVWRRVRRRV | 16 | 9 | 0.159 | 0.808 |
| | | | | NO: 4 | | | | | |
| hPAXI-7 | AMP3Ia-18 | E66 | 3 | SEQ ID | VWRRVRRVWRRVRRWVRR | 18 | 10 | 0.153 | 0.78 |
| | | | | NO: 5 | | | | | |
| hPAXI-8 | AMP3I-18 | E10 | 3 | SEQ ID | WWRRVRRVWRRVRRVVRR | 18 | 10 | 0.153 | 0.811 |
| | | | | NO: 6 | | | | | |
| hPAXI-9 | AMP3I-20 | E7 | 3 | SEQ ID | RWVRRVRRVWRRVRRVVRRW | 20 | 11 | 0.148 | 0.84 |
| | | | | NO: 7 | | | | | |
| hPAXI-10 | AMP3Ib-22 | E72 | 3 | SEQ ID | RRVWRRVRRVWRRVRRWVRRVV | 22 | 12 | 0.144 | 0.765 |
| | | | | NO: 86 | | | | | |
| hPAXI-11 | AMP3I-22 | E4 | 3 | SEQ ID | RRWVRRVRRVWRRVRRVVRRWV | 22 | 12 | 0.144 | 0.812 |
| | | | | NO: 8 | | | | | |
| hPAXI-12 | AMP3I-24 | E76 | 3 | SEQ ID | RRVWRRVRRVVRRVWRWVRRVVRR | 24 | 13 | 0.141 | 0.739 |
| | | | | NO: 9 | | | | | |
| hPAXI-13 | AMP4I-10 | E29 | 4 | SEQ ID | RRWWRRVWRW | 10 | 5 | 0.517 | 0.83 |
| | | | | NO: 10 | | | | | |
| hPAXI-14 | AMP4I-12 | E30 | 4 | SEQ ID | RRVWRWVRRWWR | 12 | 6 | 0.448 | 0.87 |
| | | | | NO: 11 | | | | | |
| hPAXI-15 | AMP4Ia-12 | E21 | 4 | SEQ ID | WWRRWRRRWRRV | 12 | 7 | 0.263 | 0.942 |
| | | | | NO: 12 | | | | | |
| hPAXI-16 | AMP4I-14 | E32 | 4 | SEQ ID | RRVWRWVRRWWRRV | 14 | 7 | 0.399 | 0.849 |
| | | | | NO: 13 | | | | | |
| hPAXI-17 | AMP4I-16 | E35 | 4 | SEQ ID | RRVWRWVRRVWRWVRR | 16 | 8 | 0.363 | 0.736 |
| | | | | NO: 14 | | | | | |
| hPAXI-18 | AMP4I-18 | E38 | 4 | SEQ ID | RRVWRWVRRVWRRVRRWV | 18 | 9 | 0.334 | 0.791 |
| | | | | NO: 15 | | | | | |
| hPAXI-19 | AMP4I-20 | E41 | 4 | SEQ ID | RRVWRWVRRVWRWVRRVVRR | 20 | 10 | 0.311 | 0.729 |
| | | | | NO: 16 | | | | | |
| hPAXI-20 | AMP4I-22 | E44 | 4 | SEQ ID | RRVWRWVRRVWRWVRRVVRRVR | 22 | 11 | 0.292 | 0.718 |
| | | | | NO: 17 | | | | | |
| hPAXI-21 | AMP4Ia-22 | E45 | 4 | SEQ ID | RRVRRWVRRVWRWVRRVWRRVR | 22 | 12 | 0.191 | 0.762 |
| | | | | NO: 18 | | | | | |
| hPAXI-22 | AMP4I-24 | E48 | 4 | SEQ ID | RRVWRWVRRVWRWVRRVVRRVRRV | 24 | 12 | 0.277 | 0.72 |
| | | | | NO: 19 | | | | | |
| hPAXI-23 | AMP4Ia-24 | E49 | 4 | SEQ ID | RRVRRWVRRVWRWVRRVWRRVRRV | 24 | 13 | 0.184 | 0.764 |
| | | | | NO: 20 | | | | | |
| hPAXI-24 | AMP5I-10 | E83 | 5 | SEQ ID | RRWWRRWWRW | 10 | 5 | 0.62 | 0.931 |
| | | | | NO: 21 | | | | | |
| hPAXI-25 | AMP5I-12 | E84 | 5 | SEQ ID | RRWWRWVRRWWR | 12 | 6 | 0.534 | 0.951 |
| | | | | NO: 22 | | | | | |
| hPAXI-26 | AMP5I-14 | E86 | 5 | SEQ ID | RRWWRWVRRWWRRV | 14 | 7 | 0.473 | 0.916 |
| | | | | NO: 23 | | | | | |
| hPAXI-27 | AMP5I-16 | E89 | 5 | SEQ ID | RRVWRWWRRVWRWVRR | 16 | 8 | 0.427 | 0.793 |
| | | | | NO: 24 | | | | | |
| hPAXI-28 | AMP5I-18 | E94 | 5 | SEQ ID | RRVWRWVRRVWRRVRRWW | 18 | 9 | 0.391 | 0.835 |
| | | | | NO: 25 | | | | | |

H, hydrophobicity and μH, hydrophobic moment were determined using the online peptide modeling software heliquest.ipmc.cnrs.fr/; peptides of the same length are minor positional variants of W; r, residues; £comprehensive name; I, 3W; II; 4W; III, 5W; h, modeled as idealized helical structures; Code, random name given to blind technicians.

TABLE 2
Primary sequences and physicochemical properties of peptides in Library 2.

| | Name in | | Trp | SEQ | | Length | | | |
|---|---|---|---|---|---|---|---|---|---|
| Name | Provisional | Code | (W) | ID NO | Sequence | (r) | Charge | H | μH |
| hPAXI-29 | AMP3IV-10 | E24 | 3 | SEQ ID | VWRRWRRRWR | 10 | 6 | 0.191 | 0.901 |
| | | | | NO: 61 | | | | | |
| hPAXI-30 | AMP3IV-12 | E61 | 3 | SEQ ID | WVRRWRRRVRRW | 12 | 7 | 0.177 | 0.89 |
| | | | | NO: 62 | | | | | |
| hPAXI-31 | AMP3IV-14 | E62 | 3 | SEQ ID | VWRRWRRVWRRVRR | 14 | 8 | 0.166 | 0.868 |
| | | | | NO: 63 | | | | | |
| hPAXI-32 | AMP3IV-16 | E65 | 3 | SEQ ID | VWRRVRRVWRRVRRRW | 16 | 9 | 0.159 | 0.827 |
| | | | | NO: 64 | | | | | |
| hPAXI-33 | AMP3IV-18 | E67 | 3 | SEQ ID | VWRRVRRVWRRVRRVWRR | 18 | 10 | 0.153 | 0.818 |
| | | | | NO: 65 | | | | | |
| hPAXI-34 | AMP3IV-20 | E68 | 3 | SEQ ID | RWVRRVRRWVRRVRRWVRRV | 20 | 11 | 0.148 | 0.82 |
| | | | | NO: 66 | | | | | |
| hPAXI-35 | AMP3IVa-20 | E69 | 3 | SEQ ID | RVWRRVRRVWRRVRRVWRRV | 20 | 11 | 0.148 | 0.822 |
| | | | | NO: 67 | | | | | |
| hPAXI-36 | AMP3IVb-20 | E70 | 3 | SEQ ID | RWVRRVRRWVRRVRRVVRRW | 20 | 11 | 0.148 | 0.844 |
| | | | | NO: 68 | | | | | |
| hPAXI-37 | AMP3IV-22 | E71 | 3 | SEQ ID | RRVWRRVRRVWRRVRRVVRRVW | 22 | 12 | 0.144 | 0.777 |
| | | | | NO: 69 | | | | | |
| hPAXI-38 | AMP3IVa-22 | E73 | 3 | SEQ ID | RRWVRRVRRWVRRVRRVVRRWV | 22 | 12 | 0.144 | 0.814 |
| | | | | NO: 70 | | | | | |
| hPAXI-39 | AMP3IVb22 | E74 | 3 | SEQ ID | RRWVRRVRRWVRRVRRWVRRVV | 22 | 12 | 0.144 | 0.791 |
| | | | | NO: 71 | | | | | |
| hPAXI-40 | AMP3IVc-22 | E75 | 3 | SEQ ID | RRVWRRVRRVWRRVRRVWRRVV | 22 | 12 | 0.144 | 0.798 |
| | | | | NO: 72 | | | | | |
| hPAXI-41 | AMP4IV-10 | E77 | 4 | SEQ ID | RRWWRRWVRW | 10 | 5 | 0.517 | 0.938 |
| | | | | NO: 73 | | | | | |
| hPAXI-42 | AMP4IV-12 | E31 | 4 | SEQ ID | RRWWRVWRRVWR | 12 | 6 | 0.448 | 0.963 |
| | | | | NO: 74 | | | | | |
| hPAXI-43 | AMP4IVa-12 | E78 | 4 | SEQ ID | VWRRWRRRWRRW | 12 | 7 | 0.263 | 0.963 |
| | | | | NO: 75 | | | | | |
| hPAXI-44 | AMP4IV-14 | E79 | 4 | SEQ ID | RRWVRWVRRWVRRW | 14 | 7 | 0.399 | 0.942 |
| | | | | NO: 76 | | | | | |
| hPAXI-45 | AMP4IV-16 | E80 | 4 | SEQ ID | RRWVRWVRRWVRWVRR | 16 | 8 | 0.363 | 0.85 |
| | | | | NO: 77 | | | | | |
| hPAXI-46 | AMP4IV-18 | E81 | 4 | SEQ ID | RRWVRWVRRWVRRVRRWV | 18 | 9 | 0.334 | 0.365 |
| | | | | NO: 78 | | | | | |
| hPAXI-47 | AMP4IV-20 | E82 | 4 | SEQ ID | RRVWRVWRRVWRVVRRVWRR | 20 | 10 | 0.311 | 0.811 |
| | | | | NO: 79 | | | | | |
| hPAXI-48 | AMP5IV-12 | E85 | 5 | SEQ ID | RRWWRVWRRWWR | 12 | 6 | 0.534 | 1.021 |
| | | | | NO: 80 | | | | | |
| hPAXI-49 | AMP5IV-14 | E88 | 5 | SEQ ID | RRWVRWWRRWVRRW | 14 | 7 | 0.473 | 1.009 |
| | | | | NO: 81 | | | | | |
| hPAXI-50 | AMP5IV-16 | E93 | 5 | SEQ ID | RRWVRWVRRWVRWWRR | 16 | 8 | 0.427 | 0.911 |
| | | | | NO: 82 | | | | | |
| hPAXI-51 | AMP5IV-18 | E95 | 5 | SEQ ID | RRVWRVWRRVWRRWRRVW | 18 | 9 | 0.391 | 0.921 |
| | | | | NO: 83 | | | | | |

H, hydrophobicity and μH, hydrophobic moment were determined using the online peptide modeling software heliquest.ipmc.cos.fr/; peptides of the same length are minor positional variants of W; r, residues; £comprehensive name; I, 3W; II; 4W; III, 5W; h, modeled as idealized helical structures; Code, random name given to blind technicians.

TABLE 3
Primary sequences and physicochemical properties of Library 3 peptides

| | Name in | | Trp | SEQ | | Length | | | |
|---|---|---|---|---|---|---|---|---|---|
| Name | Provisional | Code | (W) | ID NO | Sequence | (r) | Charge | H | μH |
| hPAXI-52 | AMP3III-12 | E20 | 3 | SEQ ID | VVRRWRRRWRRW | 12 | 7 | 0.177 | 0.904 |
| | | | | NO: 42 | | | | | |
| hPAXI-53 | AMP3III-14 | E18 | 3 | SEQ ID | WVRRWRRVVRRWRR | 14 | 8 | 0.166 | 0.914 |
| | | | | NO: 43 | | | | | |
| hPAXI-54 | AMP3III-16 | E15 | 3 | SEQ ID | VVRRWRRVVRRWRRRW | 16 | 9 | 0.159 | 0.86 |
| | | | | NO: 44 | | | | | |
| hPAXI-55 | AMP3III-18 | E12 | 3 | SEQ ID | WVRRWRRVVRRWRRVVRR | 18 | 10 | 0.153 | 0.865 |
| | | | | NO: 45 | | | | | |
| hPAXI-56 | AMP3III-20 | E9 | 3 | SEQ ID | RWVRRWRRVVRRWRRVVRRV | 20 | 11 | 0.148 | 0.864 |
| | | | | NO: 46 | | | | | |
| hPAXI-57 | AMP3III-22 | E6 | 3 | SEQ ID | RRWVRRWRRVVRRWRRVVRRVV | 22 | 12 | 0.144 | 0.834 |
| | | | | NO: 47 | | | | | |
| hPAXI-58 | AMP3III-24 | E3 | 3 | SEQ ID | RRVVRRWRRVVRRWVRVWRRVVRR | 24 | 13 | 0.141 | 0.824 |
| | | | | NO: 48 | | | | | |
| hPAXII-59 | AMP4III-12 | E22 | 4 | SEQ ID | RRWVRVWRRWWR | 12 | 6 | 0.448 | 1.002 |
| | | | | NO: 49 | | | | | |
| hPAXII-60 | AMP4IIIa-14 | E34 | 4 | SEQ ID | RRWVRVWRRWVRRW | 14 | 7 | 0.399 | 0.995 |
| | | | | NO: 51 | | | | | |
| hPAXII-61 | AMP4III-14 | E33 | 4 | SEQ ID | RRWVRVWRRWWRRV | 14 | 7 | 0.399 | 0.962 |
| | | | | NO: 50 | | | | | |
| hPAXII-62 | AMP4III-16 | E37 | 4 | SEQ ID | RRWVRVWRRWVRVWRR | 16 | 8 | 0.363 | 0.931 |
| | | | | NO: 52 | | | | | |
| hPAXII-63 | AMP4III-18 | E40 | 4 | SEQ ID | RRWVRVWRRWVRRWRRVV | 18 | 9 | 0.334 | 0.923 |
| | | | | NO: 53 | | | | | |
| hPAXII-64 | AMP4III-20 | E43 | 4 | SEQ ID | RRWVRVWRRWVRVWRRVVRR | 20 | 10 | 0.311 | 0.884 |
| | | | | NO: 54 | | | | | |
| hPAXII-65 | AMP4III-22 | E46 | 4 | SEQ ID | RRWRRVVRRWVRVWRRWVRRVR | 22 | 12 | 0.191 | 0.87 |
| | | | | NO: 55 | | | | | |
| hPAXII-66 | AMP4IIIa-22 | E47 | 4 | SEQ ID | RRWRRVVRRWVRVWRRVVRRWR | 22 | 12 | 9.191 | 0.88 |
| | | | | NO: 56 | | | | | |
| hPAXII-67 | AMP4III-24 | E50 | 4 | SEQ ID | RRWRRVVRRWVRVWRRVVRRWRRV | 24 | 13 | 0.184 | 0.873 |
| | | | | NO: 57 | | | | | |
| hPAXIII-68 | AMP5III-14 | E87 | 5 | SEQ ID | RRWVRVWRRWWRRW | 14 | 7 | 0.473 | 1.035 |
| | | | | NO: 58 | | | | | |
| hPAXIII-69 | AMP5III-16 | E92 | 5 | SEQ ID | RRWVRWWRRWVRVWRR | 16 | 8 | 0.427 | 0.955 |
| | | | | NO: 59 | | | | | |
| hPAXIII-70 | AMP5III-18 | E96 | 5 | SEQ ID | RRWVRVWRRWVRRWRRVW | 18 | 9 | 0.391 | 0.966 |
| | | | | NO: 60 | | | | | |

H, hydrophobicity and μH, hydrophobic moment were determined using the online peptide modeling software heliquest.ipmc.cnrs.fr/; peptides of the same length are minor positional variants of W; r, residues; £comprehensive name; I, 3W; II; 4W; III, 5W; b, modeled as idealized helical structures; Code, random name given to blind technicians.

TABLE 4
Primary sequences and physicochemical properties of peptides in Library 4.

| | Name in | | Trp | SEQ | | Length | | | |
|---|---|---|---|---|---|---|---|---|---|
| Name | Provisional | Code | (W) | ID NO | Sequence | (r) | Charge | H | μH |
| hPAXI-71 | E-0060 | E60 | 3 | SEQ ID | WRRRWRRW | 8 | 5 | * | * |
| | | | | NO: 26 | | | | | |
| hPAXI-72 | E-0017 | E17 | 3 | SEQ ID | VWRRVRRWVRRWRR | 14 | 8 | 0.166 | 0.851 |
| | | | | NO: 27 | | | | | |
| hPAXI-73 | E-0014 | E14 | 3 | SEQ ID | VWRRWRRWVRRVRRRV | 16 | 9 | 0.159 | 0.803 |
| | | | | NO: 28 | | | | | |
| hPAXI-74 | E-0011 | E11 | 3 | SEQ ID | VWRRVRRVVRRWRRWVRR | 18 | 10 | 0.153 | 0.798 |
| | | | | NO: 29 | | | | | |
| hPAXI-75 | E-0008 | E8 | 3 | SEQ ID | RVWRRVRRVVRRWRRWVRRV | 20 | 11 | 0.148 | 0.804 |
| | | | | NO: 30 | | | | | |
| hPAXI-76 | E-0005 | E5 | 3 | SEQ ID | RRVWRRVRRVVRRWRRWVRRVV | 22 | 12 | 0.144 | 0.779 |
| | | | | NO: 31 | | | | | |
| hPAXI-77 | E-0002 | E2 | 3 | SEQ ID | RRVVRRWRRVVRRVWRWVRRVVRR | 24 | 13 | 0.141 | 0.758 |
| | | | | NO: 32 | | | | | |
| hPAXI-78 | E2-0028 | E28 | 4 | SEQ ID | RRWWRRWW | 8 | 4 | * | * |
| | | | | NO: 33 | | | | | |
| hPAXI-79 | E-0025 | E25 | 4 | SEQ ID | WWRRWRRRWR | 10 | 6 | 0.294 | 0.959 |
| | | | | NO: 34 | | | | | |
| hPAXI-80 | E2-0036 | E36 | 4 | SEQ ID | RRWWRVVRRVVRWWRR | 16 | 8 | 0.363 | 0.819 |
| | | | | NO: 35 | | | | | |
| hPAXI-81 | E2-0039 | E39 | 4 | SEQ ID | RRWWRWVRRVVRRWRRVV | 18 | 9 | 0.334 | 0.845 |
| | | | | NO: 36 | | | | | |
| hPAXI-82 | E2-0042 | E42 | 4 | SEQ ID | RRWWRVVRRVVRWWRRVVRR | 20 | 10 | 0.795 | 0.795 |
| | | | | NO: 37 | | | | | |
| hPAXI-83 | E3-0090 | E90 | 5 | SEQ ID | RRWWRWVRRVVRWWRR | 16 | 8 | 0.427 | 0.843 |
| | | | | NO: 38 | | | | | |
| hPAXI-84 | E3-0091 | E91 | 5 | SEQ ID | RRWWRWVRRVWRWVRR | 16 | 8 | 0.427 | 0.8 |
| | | | | NO: 39 | | | | | |
| hPAXI-85 | E3-0097 | E97 | 5 | SEQ ID | RRVWRWVRRVWRRWRRWV | 18 | 9 | 0.391 | 0.848 |
| | | | | NO: 40 | | | | | |
| hPAXI-86 | E3-0098 | E98 | 5 | SEQ ID | RRWWRWWRRVVRRWRRVV | 18 | 9 | 0.391 | 0.898 |
| | | | | NO: 41 | | | | | |

H, hydrophobicity and μH, hydrophobic moment were determined using the online peptide modeling software heliquest.ipmc.cnrs.fr/; peptides of the same length are minor positional variants of W; r, residues; £comprehensive name; I, 3W; II; 4W; III, 5W; b, modeled as idealized helical structures; Code, random name given to blind technicians.

TABLE 5
Minimum inhibitory concentrations of the 13 selected peptides
with antibiotic controls corresponding to FIGS. 1-4.

| | MIC (μM) | | | | | | | | | |
|---|---|---|---|---|---|---|---|---|---|---|
| | Tob | Mero | Ceft | Oxa | Cefa | Col | E3 | E4 | E5 | E7 |
| A. baumannii | | | | | | | | | | |
| AB-A2 | 0.5 | 4 | 1 | ND | ND | 0.5 | 4 | 4 | 4 | 2 |
| AB-A3 | 16 | >16 | >16 | ND | ND | 0.25 | 2 | 4 | 4 | 4 |
| AB-F8 | >16 | 8 | >16 | ND | ND | 0.5 | 4 | 8 | 8 | 2 |
| AB-F9 | 2 | 8 | 2 | ND | ND | 0.25 | 8 | 16 | 8 | 4 |
| E. coli | | | | | | | | | | |
| Eco25922 | ND | 0.5 | 1 | ND | ND | 0.5 | 2 | 2 | 1 | 4 |
| YDC107 | ND | 1 | 4 | ND | ND | 0.5 | 0.5 | 0.5 | 0.5 | 1 |
| YDC337 | >16 | 8 | >16 | ND | ND | 0.25 | 1 | 1 | 1 | 1 |
| YDC748 | 16 | 8 | >16 | ND | ND | 0.25 | 1.5 | 1 | 3 | 1.5 |
| Enterobacter spp | | | | | | | | | | |
| EA518 | >16 | >16 | >16 | ND | ND | 0.25 | 4 | 4 | 4 | >16 |
| EC470 | 4 | >16 | >16 | ND | ND | 0.25 | 16 | 8 | 8 | 16 |
| EC560 | >16 | 16 | >16 | ND | ND | 0.25 | 8 | 8 | 8 | 16 |
| EA596 | >16 | 16 | >16 | IND | ND | 0.25 | 8 | 8 | 8 | 16 |
| K. pneumoniae | | | | | | | | | | |
| KP43816 | | 1 | 1 | ND | ND | ND | 8 | 8 | 8 | 8 |
| KPC3 | | 1 | >16 | ND | ND | 0.25 | 8 | 4 | 4 | 8 |
| KPA5 | >16 | 2 | 2 | ND | ND | >16 | 8 | 4 | 8 | 8 |
| KPB3 | >16 | 2 | 8 | ND | ND | 0.25 | 8 | 8 | 16 | 16 |
| P. aeruginosa | | | | | | | | | | |
| PA01 | 1 | ND | ND | ND | ND | 0.5 | 8 | 3 | 4 | 4 |
| PA14 | 0.5 | ND | ND | ND | ND | 0.25 | 16 | 6 | 5 | 6 |
| PA129-5 | 16 | ND | 2 | ND | ND | 0.5 | 16 | 8 | 8 | 16 |
| PA97-5 | >16 | ND | 1 | ND | ND | 0.25 | 8 | 4 | 4 | 8 |
| S. aureus | | | | | | | | | | |
| SA49775 | ND | ND | ND | 0.25 | 2 | >16 | 8 | 3 | 4 | 8 |
| SA-US300 | ND | ND | ND | 4 | 8 | >16 | 2 | 1 | 1 | 2 |
| SA18710 | ND | ND | ND | 4 | 16 | >16 | 8 | 6 | 6 | 6 |
| SA12212 | ND | ND | ND | 8 | 16 | >16 | 8 | 4 | 5 | 6 |
| E. Faecium | | | | | | | | | | |
| EF23614 | ND | ND | 8 | 0.25 | >16 | >16 | 16 | 8 | 8 | 16 |
| EF24670 | ND | ND | >16 | >16 | >16 | 0.25 | 2 | 4 | 4 | 4 |
| EF26692 | ND | ND | ND | 2 | 4 | >16 | 8 | 6 | 6 | 6 |
| EF26215 | ND | ND | ND | 2 | 4 | >16 | 4 | 8 | 8 | 4 |

| | MIC (μM) | | | | | | | | |
|---|---|---|---|---|---|---|---|---|---|
| | E32 | E35 | E68 | E69 | E70 | E71 | E72 | E75 | E80 |
| A. baumannii | | | | | | | | | |
| AB-A2 | 4 | 4 | 4 | 8 | 4 | 8 | 4 | 2 | 16 |
| AB-A3 | 4 | 4 | 4 | 8 | 8 | 8 | 2 | 2 | 16 |
| AB-F8 | 8 | 4 | 8 | 8 | 8 | 8 | 4 | 2 | 16 |
| AB-F9 | 16 | 4 | 4 | 8 | 4 | 8 | 4 | 4 | 16 |
| E. coli | | | | | | | | | |
| Eco25922 | 4 | 2 | 4 | 4 | 2 | 1 | 2 | 2 | 8 |
| YDC107 | 2 | 3 | 4 | 4 | 4 | 8 | 2 | 2 | 2 |
| YDC337 | 1 | 2 | 1 | 2 | 2 | 2 | 2 | 0.5 | 2 |
| YDC748 | 1.5 | 0.5 | 2 | 0.5 | 0.5 | 0.5 | 0.5 | 0.5 | 2 |
| Enterobacter spp | | | | | | | | | |
| EA518 | 16 | 8 | 16 | >16 | 16 | 8 | 4 | 16 | >16 |
| EC470 | 8 | 4 | 16 | 16 | 16 | 8 | 16 | 16 | 8 |
| EC560 | 4 | 4 | 4 | 8 | 8 | 4 | 8 | 4 | 8 |
| EA596 | 4 | 4 | 8 | 8 | 8 | 2 | 4 | 2 | 2 |
| K. pneumoniae | | | | | | | | | |
| KP43816 | 4 | 4 | 8 | 16 | 8 | 8 | 8 | 8 | 8 |
| KPC3 | 2 | 2 | 16 | 16 | 8 | 8 | 16 | 16 | 16 |
| KPA5 | 4 | 2 | 2 | 2 | 2 | 2 | 1 | 2 | 4 |
| KPB3 | 1 | 2 | 0.25 | 0.25 | 0.5 | 0.25 | 0.5 | 4 | 0.5 |
| P. aeruginosa | | | | | | | | | |
| PA01 | 8 | 6 | 8 | 8 | 8 | 8 | 8 | 8 | >16 |
| PA14 | 16 | 8 | 4 | 4 | 4 | 4 | 4 | 4 | 16 |
| PA129-5 | 8 | 8 | 3 | 4 | 4 | 2 | 3 | 2.5 | 6 |
| PA97-5 | 16 | 8 | 8 | 2 | 2.13 | 4 | 2.25 | 13 | 2 |
| S. aureus | | | | | | | | | |
| SA49775 | 3 | 1.5 | 1 | 2 | 2 | 2 | 1 | 2 | 2 |
| SA-US300 | 2 | 1 | 1 | 4 | 4 | 2 | 2 | 2 | 1 |
| SA18710 | 6 | 5 | 2 | 4 | 2 | 1 | 2 | 2 | 4 |
| SA12212 | 6 | 4 | 8 | 8 | 4 | 1 | 2 | 4 | 1 |
| E. Faecium | | | | | | | | | |
| EF23614 | >16 | 4 | 16 | 16 | 16 | 8 | >16 | 8 | >16 |
| EF24670 | 16 | 2 | 0.5 | 2 | 0.5 | 1 | 1 | 1 | 1 |
| EF26692 | 6 | 2 | 2 | 2 | 1 | 1 | 1 | 1 | 1 |
| EF26215 | 8 | 0.5 | 0.5 | 0.5 | 1 | 0.5 | 1 | 0.5 | 0.5 |

ND, not determined;
Enterobacter spp: EA (aerogenes), EC (cloacae), E. aerogenes;
E. Faecium, Enterococcus Faecium;
clinical strains are from University of Pittsburgh Medical Center (UPMC)
Gray highlight, colistin resistance, breakpoint 2 mg/L or ~2 μM [26-28];
Tob, tobramycin;
Mero, meropenem;
Ceft, ceftazidime;
Oxa, oxacillin;
Cefa, cefazolin;
Col, colistin

TABLE 7
Minimum inhibitory concentrations of the 13 selected peptides and colistin corresponding to FIG. 5.

| | MIC, μM | | | | | | | | | | | | | |
|---|---|---|---|---|---|---|---|---|---|---|---|---|---|---|
| | Colistin | E3 | E4 | E5 | E7 | E32 | E35 | E68 | E69 | E70 | E71 | E72 | E75 | E80 |
| A. baumannii | | | | | | | | | | | | | | |
| AB273 | 0.5 | 2 | 1 | 1 | 4 | 2 | 2 | 2 | 2 | 2 | 2 | 2 | 2 | 2 |
| AB274 | 0.25 | 1 | 0.5 | 0.5 | 2 | 8 | 0.5 | 0.5 | 1 | 1 | 0.5 | 0.5 | 0.5 | 0.5 |
| AB275 | 0.25 | 4 | 4 | 16 | 16 | 4 | 4 | 2 | 2 | 2 | 2 | 2 | 2 | 4 |
| AB275 | 0.5 | 2 | 2 | 1 | 2 | 2 | 2 | 0.5 | 0.5 | 0.5 | 0.5 | 0.5 | 2 | 2 |
| E. coli | | | | | | | | | | | | | | |
| Eco546 | 0.25 | 0.25 | 0.25 | 1 | 0.25 | 0.5 | 0.25 | 0.5 | 0.5 | 0.5 | 1 | 0.5 | 4 | 1 |
| Eco549 | 1 | 0.5 | 0.5 | 1 | 1 | 1 | 0.5 | 1 | 0.5 | 0.5 | 0.5 | 0.5 | 4 | 1 |
| Eco541 | 1 | 1 | 0.25 | 1 | 2 | 0.5 | 0.5 | 0.5 | 0.5 | 0.5 | 0.5 | 0.5 | 1 | 1 |
| Eco543 | 8 | 2 | 0.5 | 4 | 2 | 1 | 1 | 2 | 1 | 1 | 1 | 0.5 | 8 | 2 |
| Enterobacter spp | | | | | | | | | | | | | | |
| EC544 | 4 | >16 | 2 | 2 | 4 | >16 | 8 | 1 | 2 | 2 | 2 | 4 | 2 | 2 |
| EC545 | 2 | 4 | 1 | 1 | 4 | 4 | 1 | 1 | 2 | 2 | 1 | 1 | 2 | 2 |
| EA62 | 2 | >16 | 4 | 8 | 16 | >16 | 16 | 4 | 8 | 8 | 8 | 4 | 8 | 16 |
| EA547 | 0.25 | 8 | 2 | 4 | 8 | 16 | 4 | 4 | 4 | 4 | 2 | 4 | 2 | 16 |
| K. pneumoniae | | | | | | | | | | | | | | |
| KP550 | 0.25 | 16 | 2 | 4 | 16 | >16 | 1 | 16 | 8 | 8 | 2 | 2 | 2 | 8 |
| KP542 | >16 | 16 | 8 | 16 | >16 | >16 | >16 | 2 | 8 | 4 | 8 | >16 | 16 | >16 |
| KP548 | 0.25 | 8 | 2 | 2 | 8 | >16 | 2 | 4 | 4 | 4 | 2 | 2 | 2 | 8 |
| KP552 | 0.25 | >16 | 4 | 8 | 16 | 16 | 2 | 4 | 16 | 8 | 4 | 4 | 8 | >16 |
| P. aeruginosa | | | | | | | | | | | | | | |
| PA229 | >16 | 4 | 4 | 4 | 2 | 16 | 16 | 4 | 16 | 1 | 1 | 8 | 16 | >16 |
| PA231 | 1 | 16 | 4 | 8 | 16 | >16 | >16 | 8 | 8 | 8 | 8 | 4 | 16 | >16 |
| PA216 | 1 | 8 | 2 | 2 | 4 | 8 | 4 | 4 | 8 | 2 | 2 | 2 | 4 | 8 |
| PA230 | 1 | 16 | 4 | 2 | 8 | 1 | 2 | 2 | 0 | 1 | 2 | 2 | 2 | 4 |
| S. aureus | | | | | | | | | | | | | | |
| SA461 | >16 | 4 | 0.5 | 0.5 | 2 | 2 | 0.5 | 1 | 2 | 1 | 1 | 0.5 | 1 | 2 |
| SA561 | >16 | 16 | 4 | 4 | 16 | 4 | 1 | 2 | 8 | 4 | 8 | 2 | 8 | 8 |
| SA462 | >16 | 16 | 4 | 1 | 8 | 4 | 1 | 4 | 8 | 8 | 8 | 2 | 1 | 8 |
| SA463 | >16 | 2 | 0.5 | 0.5 | 0.5 | 0.5 | 0.5 | 2 | 2 | 2 | 2 | 2 | 1 | 8 |
| E. Faecium | | | | | | | | | | | | | | |
| EF673 | >16 | 2 | 2 | 4 | 4 | 16 | 4 | 4 | 2 | 2 | 2 | 4 | 2 | 4 |
| EF500 | >16 | 1 | 1 | 1 | 2 | 1 | 1 | 1 | 1 | 1 | 1 | 1 | 1 | 1 |
| EF672 | >16 | 8 | 4 | 4 | 8 | 16 | 4 | 1 | 1 | 1 | 1 | 4 | 2 | 4 |
| EF671 | >16 | 4 | 2 | 2 | 2 | 4 | 2 | 1 | 2 | 2 | 2 | 4 | 2 | 4 |

NA, not applicable;
clinical strains re from the CDC;
Enterobacter spp: EA (aerogenes), EC (cloacae), E. aerogenes;
E. Facium, Enterococcus Facium;
Gray highlight, gram-negative colistin resistance, breakpoint 2 mg/L or ~2 μM [26-28]

TABLE 8
PAX activity against colistin-resistant
CDC AR isolates, not included in Tables 5-7.
*MIC, μM

| | E4 | E5 | E35 | E72 | E70 | colistin |
|---|---|---|---|---|---|---|
| KP1010 | 4 | 2 | 8 | 1 | 1 | 16 |
| KP87 | 4 | 2 | 8 | 1 | 1 | >16 |
| KP507 | 4 | 4 | 4 | 4 | 2 | 16 |
| KP1045 | 4 | 4 | 8 | 4 | 2 | >16 |
| KP1041 | 4 | 4 | 2 | 4 | 4 | >16 |
| KP46 | 2 | 2 | 4 | 2 | 2 | 4 |
| KP125 | 2 | 2 | 2 | 2 | 8 | 8 |
| AB288 | 1 | 0.5 | 2 | 1 | 1 | 1^{a} |
| AB300 | 2 | 1 | 2 | 0.5 | 0.5 | 1^{a} |
| AB301 | 2 | 2 | 2 | 2 | 1 | 1^{a} |
| AB303 | 1 | 2 | 1 | 4 | 2 | 0.5^{a} |

*Break point for resistance to colistin is 2 μg/mL or ~2 μM [26-28]
^{a}Resistance not confirmed; resistance to PAX, MIC > 8 μM,
KP, klebsiella pneumoniae;
AB, Acinetobacter baumannii

TABLE 9
Peptide sequences
Position 1
3-TRP residues

| | | | |
|---|---|---|---|
| 3-TRP | AMP 3I-10 | E-0023 | WWRRVRRRWR (SEQ ID NO: 1) |
| | AMP 3I-12 | E-0019 | WWRRVRRRWRRV (SEQ ID NO: 2) |
| | AMP 3Ia-14 | E-0063 | VWRRVRRWWRRVRR (SEQ ID NO: 84) |
| | AMP 3I-14 | E-0016 | WWRRVRRVWRRVRR (SEQ ID NO: 3) |
| | AMP 3Ia-16 | E-0064 | VWRRVRRWWRRVRRRV (SEQ ID NO: 85) |
| | AMP 3I-16 | E-0013 | WWRRVRRVWRRVRRRV (SEQ ID NO: 4) |
| | AMP 3Ia-18 | E-0066 | VWRRVRRVWRRVRRWVRR (SEQ ID NO: 5) |
| | 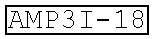 | E-0010 | WWRRVRRVWRRVRRVVRR (SEQ ID NO: 6) |
| | AMP 3I-20 | E-0007 | RWVRRVRRVWRRVRRVVRRW (SEQ ID NO: 7) |
| | AMP 3Ib-22 | E-0072 | RRVWRRVRRVWRRVRRWVRRVV (SEQ ID NO: 86) |
| | AMP 3I-22 | E-0004 | RRWVRRVRRVWRRVRRVVRRWV (SEQ ID NO: 8) |
| | AMP 3I-24 | E-0076* | RRVWRRVRRVVRRVWRWVRRVVRR (SEQ ID NO: 9) |
| 4 Trp | AMP 4I-10 | E2-0029 | RRWWRRVWRW (SEQ ID NO: 10) |
| | AMP 4I-12 | E2-0030 | RRVWRWVRRWWR (SEQ ID NO: 11) |
| | AMP 4I-14 | E2-0032 | RRVWRWVRRWWRRV (SEQ ID NO: 13) |
| | AMP 4I-16 | E2-0035 | RRVWRWVRRVWRWVRR (SEQ ID NO: 14) |
| | AMP 4I-18 | E2-0038 | RRVWRWVRRVWRRVRRWV (SEQ ID NO: 15) |
| | AMP 4I-20 | E2-0041 | RRVWRWVRRVWRWVRRVVRR (SEQ ID NO: 16) |
| | AMP 4Ia-22 | E2-0044 | RRVWRWVRRVWRWVRRVVRRVR (SEQ ID NO: 17) |
| | AMP 4Ib-22 | E2-0045 | RRVRRWVRRVWRWVRRVWRRVR (SEQ ID NO: 18) |
| | AMP 4I-24 | E2-0048 | RRVWRWVRRVWRWVRRVVRRVRRV (SEQ ID NO: 19) |
| | AMP 4Ib-24 | E2-0049 | RRVRRWVRRVWRWVRRVWRRVRRV (SEQ ID NO: 20) |
| 5 Trp | AMP 5-10 | E3-0083 | RRWWRRWWRW (SEQ ID NO: 21) |
| | AMP 5I-12 | E3-0084 | RRWWRWVRRWWR (SEQ ID NO: 22) |
| | AMP 5I-14 | E3-0086 | RRWWRWVRRWWRRV (SEQ ID NO: 23) |
| | AMP 5I-16 | E3-0089 | RRVWRWWRRVWRWVRR (SEQ ID NO: 24) |
| | AMP 5I-18 | E3-0094 | RRVWRWVRRVWRRVRRWW (SEQ ID NO: 25) |

Position II

| | | |
|---|---|---|
| AMP 3II-8 | E-0060 | WRRRWRRW (SEQ ID NO: 26) |
| AMP 3II-14 | E-0017 | VWRRVRRWVRRWRR (SEQ ID NO: 27) |
| AMP 3II-16 | E-0014 | VWRRWRRWVRRVRRRV (SEQ ID NO: 28) |
| AMP 3II-18 | E-0011 | VWRRVRRVVRRWRRWVRR (SEQ ID NO: 29) |
| AMP 3II-20 | E-0008 | RVWRRVRRVVRRWRRWVRRV (SEQ ID NO: 30) |
| AMP 3II-22 | E-0005 | RRVWRRVRRVVRRWRRWVRRVV (SEQ ID NO: 31) |
| AMP 3IIb-24 | E-0002 | RRVVRRWRRVVRRVWRWVRRVVRR (SEQ ID NO: 32) |
| AMP 3IIa-24 | E-0001 | RRWWRRVRRVVRRVVRVVRRWVRR24 (SEQ ID NO: 87) |
| AMP 4-8 | E2-0028 | RRWWRRWW (SEQ ID NO: 33) |
| AMP 4-10 | E-0025 | WWRRWRRRWR (SEQ ID NO: 34) |
| AMP 4II-16 | E2-0036 | RRWWRVVRRVVRWWRR (SEQ ID NO: 35) |
| AMP 4II-18 | E2-0039 | RRWWRWVRRVVRRWRRVV (SEQ ID NO: 36) |
| AMP 4II-20 | E2-0042 | RRWWRVVRRVVRWWRRVVRR (SEQ ID NO: 37) |
| AMP 5-10 | E3-0083 | RRWWRRWWRW (SEQ ID NO: 21) |
| AMP-5IIa-16 | E3-0090 | RRWWRWVRRVVRWWRR (SEQ ID NO: 38) |
| AMP-5IIb-16 | E3-0091 | RRWWRWVRRVWRWVRR (SEQ ID NO: 39) |
| AMP-5IIa-18 | E3-0097 | RRVWRWVRRVWRRWRRWV (SEQ ID NO: 40) |
| AMP-5IIb-18 | E3-0098 | RRWWRWWRRVVRRWRRVV (SEQ ID NO: 41) |

Position III

| | | |
|---|---|---|
| AMP 3III-12 | E-0020 | VVRRWRRRWRRW (SEQ ID NO: 42) |
| AMP 3III-14 | E-0018 | WVRRWRRVVRRWRR (SEQ ID NO: 43) |
| AMP 3III-16 | E-0015 | VVRRWRRVVRRWRRRW (SEQ ID NO: 44) |
| AMP 3III-18 | E-0012 | WVRRWRRVVRRWRRVVRR (SEQ ID NO: 45) |
| AMP 3III-20 | E-0009 | RWVRRWRRVVRRWRRVVRRV (SEQ ID NO: 46) |
| AMP 3III-22 | E-0006 | RRWVRRWRRVVRRWRRVVRRVV (SEQ ID NO: 47) |
| AMP 3III-24 | E-0003 | RRVVRRWRRVVRRWVRVWRRVVRR (SEQ ID NO: 48) |
| | 4 Trp | |
| AMP 4IIIa-12 | E-0022 | VWRRWRRRWRRW (SEQ ID NO: 75) |
| AMP 4IIIa-14 | E2-0033 | RRWVRVWRRWWRRV (SEQ ID NO: 50) |
| AMP 4IIIb-14 | E2-0034 | RRWVRVWRRWVRRW (SEQ ID NO: 51) |
| AMP 4IIIC-14 | E2-0033 | RRWVRVWRRWWRRV (SEQ ID NO: 50) |
| AMP 4III-16 | E2-0037 | RRWVRVWRRWVRVWRR (SEQ ID NO: 52) |
| AMP 4III-18 | E2-0040 | RRWVRVWRRWVRRWRRVV (SEQ ID NO: 53) |
| AMP 4III-20 | E2-0043 | RRWVRVWRRWVRVWRRVVRR (SEQ ID NO: 54) |
| AMP 4IIIa-22 | E2-0046 | RRWRRVVRRWVRVWRRWVRRVR (SEQ ID NO: 55) |
| AMP 4IIIb-22 | E2-0047 | RRWRRVVRRWVRVWRRVVRRWR (SEQ ID NO: 56) |
| AMP 4III-24 | E2-0050 | RRWRRVVRRWVRVWRRVVRRWRRV (SEQ ID NO: 57) |
| AMP 5III-14 | E3-0087 | RRWVRVWRRWWRRW (SEQ ID NO: 58) |
| AMP 5III-16 | E3-0092 | RRWVRWWRRWVRVWRR (SEQ ID NO: 59) |
| AMP 5III-18 | E3-0096 | RRWVRVWRRWVRRWRRVW (SEQ ID NO: 60) |

Position IV

| | | |
|---|---|---|
| AMP 3IV-10 | E-0024 | VWRRWRRRWR (SEQ ID NO: 61) |
| AMP 3IV-12 | E-0061 | WVRRWRRRVRRW (SEQ ID NO: 62) |
| AMP 3IV-14 | E-0062 | VWRRWRRVWRRVRR (SEQ ID NO: 63) |
| AMP 3IV-16 | E-0065 | VWRRVRRVWRRVRRRW (SEQ ID NO: 64) |
| AMP 3IV-18 | E-0067 | VWRRVRRVWRRVRRVWRR (SEQ ID NO: 65) |
| AMP 3IV-20 | E-0069 | RVWRRVRRVWRRVRRVWRRV (SEQ ID NO: 67) |
| AMP 3Iva-20 | E-0068 | RWVRRVRRWVRRVRRWVRRV (SEQ ID NO: 66) |
| AMP 3IVb-20 | E-0070 | RWVRRVRRWVRRVRRVVRRW (SEQ ID NO: 68) |
| AMP 3IV-22 | E-0075 | RRVWRRVRRVWRRVRRVWRRVV (SEQ ID NO: 72) |
| AMP 3IVa-22 | E-0074 | RRWVRRVRRWVRRVRRWVRRVV (SEQ ID NO: 71) |
| AMP 3IVb-22 | E-0073 | RRWVRRVRRWVRRVRRVVRRWV (SEQ ID NO: 70) |
| AMP 3IVc-22 | E-0071 | RRVWRRVRRVWRRVRRVVRRVW (SEQ ID NO: 69) |
| AMP 4IV-10 | E2-0077 | RRWWRRWVRW (SEQ ID NO: 73) |
| AMP 4IVa-12 | E-0021 | WWRRWRRRWRRV (SEQ ID NO: 12) |
| AMP 4IVb-12 | E-0022 | VWRRWRRRWRRW (SEQ ID NO: 75) |
| AMP 4IVc-12 | E2-0078 | RRWWRVWRRVWR (SEQ ID NO: 74) |
| AMP 4IV-14 | E2-0079 | RRWVRWVRRWVRRW (SEQ ID NO: 76) |
| AMP 4IV-16 | E2-0080 | RRWVRWVRRWVRWVRR (SEQ ID NO: 77) |
| AMP 4IV-18 | E2-0081 | RRWVRWVRRWVRRVRRWV (SEQ ID NO: 78) |
| AMP 4IV-20 | E2-0082 | RRVWRVWRRVWRVVRRVWRR (SEQ ID NO: 79) |
| AMP 5IV-12 | E3-0085 | RRWWRVWRRWWR (SEQ ID NO: 80) |
| AMP 5IV-14 | E3-0088 | RRWVRWWRRWVRRW (SEQ ID NO: 81) |
| AMP 5IV-16 | E3-0093 | RRWVRWVRRWVRWWRR (SEQ ID NO: 82) |
| AMP 5IV-18 | E3-0095 | RRVWRVWRRVWRRWRRVW (SEQ ID NO: 83) |

1. A composition comprising an antimicrobial cationic amphipathic polypeptide (PAX), wherein the PAX comprises a sequence of one of SEQ ID NOs: 1-87 or a sequence having at least 85% sequence identity with one of SEQ ID NOs: 1-87.

2. The composition of claim 1, wherein the PAX comprises a sequence selected from the group consisting of SEQ ID NO: 48, SEQ ID NO: 8, SEQ ID NO: 31, SEQ ID NO: 7, SEQ ID NO: 14, SEQ ID NO: 66, SEQ ID NO: 67, SEQ ID NO: 68, SEQ ID NO: 69, SEQ ID NO: 86, SEQ ID NO: 72, and SEQ ID NO: 77.

3. The composition of claim 1, wherein the PAX is 20 to 24 amino acids in length and comprises a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 86 and SEQ ID NO: 87.

4. The composition of claim 1, wherein the PAX is 16 amino acids in length and comprises a sequence selected from the group consisting of SEQ ID NO: 14, SEQ ID NO: 35, SEQ ID NO: 52 and SEQ ID NO: 77.

5. The composition of claim 4, wherein the PAX comprises SEQ ID NO: 14.

6. The composition of claim 1, wherein the PAX is 14 to 18 amino acids in length and comprises a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83.

7. The composition of claim 1, wherein the PAX comprises a sequence selected from the group consisting of SEQ ID NO: 7, SEQ ID NO: 8, SEQ ID NO: 9, SEQ ID NO: 14, SEQ ID NO: 23, SEQ ID NO: 24, SEQ ID NO: 25 and SEQ ID NO: 86.

8. The composition of claim 1, wherein the PAX comprises a sequence selected from the group consisting of SEQ ID NO: 30, SEQ ID NO: 31, SEQ ID NO: 32, SEQ ID NO: 35, SEQ ID NO: 38, SEQ ID NO: 39, SEQ ID NO: 40 and SEQ ID NO: 41.

9. The composition of claim 1, wherein the PAX comprises a sequence selected from the group consisting of SEQ ID NO: 46, SEQ ID NO: 47, SEQ ID NO: 48, SEQ ID NO: 52, SEQ ID NO: 58, SEQ ID NO: 59 and SEQ ID NO: 60.

10. The composition of claim 1, wherein the PAX comprises a sequence selected from the group consisting of SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 77, SEQ ID NO: 81, SEQ ID NO: 82 and SEQ ID NO: 83.

11. The composition of claim 1, wherein the PAX is between 8 and 24 amino acids in length.

12. A method of treating a microbial infection in a subject in need thereof comprising administering to the subject a therapeutically effective amount of a composition comprising an antimicrobial cationic amphipathic polypeptide (PAX) comprising a sequence of one of SEQ ID NOs: 1-87.

13. The method of claim 12, wherein the PAX comprises a sequence selected from the group consisting of SEQ ID NO: 48, SEQ ID NO: 8, SEQ ID NO: 31, SEQ ID NO: 7, SEQ ID NO: 14, SEQ ID NO: 66, SEQ ID NO: 67, SEQ ID NO: 68, SEQ ID NO: 69, SEQ ID NO: 86, SEQ ID NO: 72, and SEQ ID NO: 77.

14. The method of claim 12, wherein the PAX is 20 to 24 amino acids in length and comprises a sequence selected from the group consisting of SEQ ID NO: 7, SEQ ID NO: 8, SEQ ID NO: 9, SEQ ID NO: 25, SEQ ID NO: 30, SEQ ID NO: 31, SEQ ID NO: 32, SEQ ID NO: 46, SEQ ID NO: 47, SEQ ID NO: 48, SEQ ID NO: 69, SEQ ID NO: 70, SEQ ID NO: 71, SEQ ID NO: 72, SEQ ID NO: 86 and SEQ ID NO: 87.

15. The method of claim 12, wherein the PAX is 16 amino acids in length and comprises a sequence selected from the group consisting of SEQ ID NO: 14, SEQ ID NO: 35, SEQ ID NO: 52 and SEQ ID NO: 77.

16. The method of claim 15, wherein the PAX comprises SEQ ID NO: 14.

17. The method of claim 12, wherein the PAX is 14 to 18 amino acids in length and comprises a sequence selected from the group consisting of SEQ ID NO: 23, SEQ ID NO: 24, SEQ ID NO: 38, SEQ ID NO: 39, SEQ ID NO: 40, SEQ ID NO: 41, SEQ ID NO: 58, SEQ ID NO: 59, SEQ ID NO: 60, SEQ ID NO: 81, SEQ ID NO: 82 and SEQ ID NO: 83.

18. The method of claim 12, wherein the PAX comprises a sequence selected from the group consisting of SEQ ID NO: 7, SEQ ID NO: 8, SEQ ID NO: 9, SEQ ID NO: 14, SEQ ID NO: 23, SEQ ID NO: 24, SEQ ID NO: 25 and SEQ ID NO: 86.

19. The method of claim 12, wherein the PAX comprises a sequence selected from the group consisting of SEQ ID NO: 30, SEQ ID NO: 31, SEQ ID NO: 32, SEQ ID NO: 35, SEQ ID NO: 38, SEQ ID NO: 39, SEQ ID NO: 40 and SEQ ID NO: 41.

20. The method of claim 12, wherein the PAX comprises a sequence selected from the group consisting of SEQ ID NO: 46, SEQ ID NO: 47, SEQ ID NO: 48, SEQ ID NO: 52, SEQ ID NO: 58, SEQ ID NO: 59 and SEQ ID NO: 60.

21. The method of claim 12, wherein the PAX comprises a sequence selected from the group consisting of SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 77, SEQ ID NO: 81, SEQ ID NO: 82 and SEQ ID NO: 83.

22. The method of claim 12, wherein the PAX is between 8 and 24 amino acids in length.

23. The method of claim 12, wherein the microbial infection is a bacterial infection.

24. The method of claim 23, wherein the bacteria is Enterococcus faecium, Staphylococcus aureus, Klebsiella pneumoniae, Acinetobacter baumannii, Pseudomonas aeruginosa, Escherichia coli, or Enterobacter spp.

25. The method of claim 23, wherein the bacteria is Escherichia coli, Pseudomonas aeruginosa, or Klebsiella pneumoniae.

26. The method of claim 23, wherein the bacteria is Acinetobacter, Candida auris, Clostridioides difficile, Enterobacteriaceae, Neisseria gonorrhoeae, Campylobacter, ESBL-producing Enterobacteriaceae, Vancomycin-resistant Enterococcus (VRE), Pseudomonas aeruginosa, nontyphoidal Salmonella, Salmonella serotype Typhi, Shigella, Methicillin-resistant Staphylococcus aureus (S. aureus) (MRSA), Streptococcus pneumoniae, Tuberculosis, resistant Group A Streptococcus, resistant Group B Streptococcus, Mycoplasma genitalium, or Bordetella pertussis.

27. The method of claim 23, wherein the bacteria is drug resistant.

28. The method of claim 23, wherein the bacteria is resistant to one or more of tobramycin, meropenem, ceftazidime, oxacillin, cefazolin, and colistin.

29. The method of claim 12, wherein the PAX is administered to the subject at a dosage from about 1 mg/kg body weight to about 100 mg/kg body weight.

30. The method of claim 12, wherein the PAX is administered at a dosage from about 5 mg/kg body weight to about 30 mg/kg body weight.
